(19) United States
(12) Patent Application Publication
(10) Pub. No.: US 2011/0319954 A1
(43) Pub. Date: Dec. 29, 2011

(54) METRICS AND TECHNIQUES FOR OPTIMIZATION OF CARDIAC THERAPIES

(75) Inventors: Imran Niazi, Milwaukee, WI (US); Kyungmoo Ryu, Palmdale, CA (US); Peter Chelius, Franklin, WI (US); Scott Patrick Simon, Billings, MT (US); Michael Kane, Delafield, WI (US); Timothy A. Fayram, Gilroy, CA (US)

(73) Assignee: PACESETTER, INC., Sylmar, CA (US)

(21) Appl. No.: 12/825,063

(22) Filed: Jun. 28, 2010

(51) Int. Cl. A61N 1/00

(52) U.S. Cl. 607/17

(57) ABSTRACT

An exemplary method includes, based on metrics available as input to a chronic phase optimization algorithm for selecting an optimal electrode configuration for delivery of a cardiac pacing therapy, executing the chronic phase optimization algorithm during an acute phase to select an optimal electrode configuration for delivery of a cardiac pacing therapy; during the acute phase, acquiring position information with respect to time for electrodes implanted in a body; determining one or more acute phase metrics based on the acquired position information; and validating the chronic phase optimization algorithm based at least in part on the one or more acute phase metrics.

### TECHNICAL FIELD

Subject matter presented herein relates generally to cardiac therapies (e.g., cardiac pacing therapies, cardiac stimulation therapies, etc.) and techniques to optimize such therapies.

### BACKGROUND

Cardiac resynchronization therapy (CRT) provides an electrical solution to the symptoms and other difficulties brought on by heart failure (HF). CRT can call for delivery of electrical stimuli to the heart in a manner that synchronizes contraction and enhances performance. When CRT delivers stimuli to the right and left ventricles, this is called biventricular pacing. Biventricular pacing aims to improve efficiency of each contraction of the heart and the amount of blood pumped to the body. This helps to lessen the symptoms of heart failure and, in many cases, helps to stop the progression of the disease.

CRT is typically administered via an implantable device such as a pacemaker (e.g., called a CRT-P) or an ICD that has a built-in pacemaker (e.g., called a CRT-D). A CRT-D has the added ability to defibrillate the heart if a patient is at risk for life-threatening arrhythmias. Most traditional ICDs or pacemakers have either one lead placed in the heart's right atrium (RA) or the heart's right ventricle (RV) or two leads where one is placed in the heart's RA and the other is placed in the heart's RV. CRT devices typically have three leads; one placed in the RA, one placed in the RV and one placed in a vein along the left ventricle (LV). Such a configuration allows for bi-ventricular pacing.

Some CRT devices are configured to connect to leads that have series of electrodes that can allow for more optimal delivery of pacing stimuli than leads with few electrodes (e.g., a lead with a tip electrode and a neighboring ring electrode). For example, a CRT platform developed by Pacesetter, Inc. (dba St. Jude Medical Cardiac Rhythm Management Division, Sylmar, Calif.) is configured for use with a LV lead having a quartet of LV electrodes. The St. Jude platform also includes a programmed optimization algorithm marketed as the QUICKOPT® algorithm that can acquire data and optimize CRT based on the acquired data. A next generation QUICKOPT® algorithm, referred to herein as “QuickStim”, contemplates a pacing optimization algorithm that selects an optimal single or optimal multisite pacing settings for implantable pacing devices, with multiple LV pacing capability and a multipolar LV lead.

CRT can improve a variety of cardiac performance measures including left ventricular mechanical function, cardiac index, decreased pulmonary artery pressures, decrease in myocardial oxygen consumption, decrease in dynamic mitral regurgitation, increase in global ejection fraction, decrease in NYHA class, increased quality of life scores, increased distance covered during a 6-minute walk test, etc. Effects such as reverse modeling may also be seen, for example, three to six months after initiating CRT. Patients that show such improvements are classified as CRT “responders”. However, for a variety of reasons, not all patients respond to CRT. For example, if a left ventricular stimulation lead cannot locate an electrode in a favorable position, then a patient may not respond to CRT.

Often, the ability to respond and the extent of response to CRT depends on an initial set-up of a CRT device in a patient. As described herein, various exemplary technologies aim to improve a clinician's ability to set-up a CRT at implant and to optionally optimize thereafter. Various exemplary techniques include metrics that may be based in part on information acquired from a localization system.

### SUMMARY

An exemplary method includes, based on metrics available as input to a chronic phase optimization algorithm for selecting an optimal electrode configuration for delivery of a cardiac pacing therapy, executing the chronic phase optimization algorithm during an acute phase to select an optimal electrode configuration for delivery of a cardiac pacing therapy; during the acute phase, acquiring position information with respect to time for electrodes implanted in a body; determining one or more acute phase metrics based on the acquired position information; and validating the chronic phase optimization algorithm based at least in part on the one or more acute phase metrics. Various other methods, devices, systems, etc., are also disclosed.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for sensing and/or delivering stimulation and/or shock therapy. Other devices with more or fewer leads may also be suitable.

FIG. 2 is a functional block diagram of an exemplary implantable stimulation device illustrating basic elements that are configured to provide cardioversion, defibrillation, pacing stimulation and/or other tissue stimulation. The implantable stimulation device is further configured to sense information and administer therapy responsive to such information.

FIG. 3 is a block diagram of an exemplary method for selecting one or more configurations and optimizing therapy based at least in part on one or more metrics.

FIG. 4 is a block diagram of an exemplary method for optimizing a therapy based on one or more metrics such as vector metrics.

FIG. 5 is an exemplary arrangement of a lead and electrodes for acquiring position information and optionally other information for use in determining one or more metrics.

FIG. 6 is a diagram of exemplary vectors and relationships between electrodes and a block diagram of exemplary metrics that may be used to select one or more of the electrodes.

FIG. 7 is a series of vector waveform and ECG plots that may be used to determine one or more vector metrics.

FIG. 8 is a diagram of an exemplary area metric and a series of area waveform plots that may be used to determine one or more area metrics.

FIG. 9 is a series of three-dimensional plots of volume metrics, including a local volume estimator metric and a regional volume estimator metric.

FIG. 10 is a series of waveform and IEGM plots suitable for determining one or more mechanical dyssynchrony metrics.

FIG. 11 is a series of plots suitable for determining electrical activation and mechanical activation metrics.

FIG. 12 is a series of septal and lateral wall motion plots along with ECG plots suitable for calculating various metrics.

FIG. 13 is a block diagram of various exemplary metrics that may be used to select or optimize electrode configuration for delivery of a cardiac therapy.

FIG. 14 is a block diagram of an exemplary method for validating an optimization algorithm for implementation in a chronic phase.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators are typically used to reference like parts or elements throughout.

### Overview

Various exemplary techniques described herein pertain to optimization of electrode configurations for delivery of pacing therapies such as CRT. Various methods pertain to validation of an optimization algorithm for chronic use by an implantable device. Such a method aims to validate the algorithm during an acute phase, for example, where equipment is available to acquire data that an implantable device may not be able to acquire. For example, position versus time data for various electrodes may be acquired by a localization system in an acute or intraoperative phase and relied on to validate performance of an optimization algorithm destined for chronic use (e.g., implementation by an implantable device, optionally in conjunction with a programming device or programmer for the implantable device).

### Exemplary Stimulation Device

Various techniques described below may be implemented in connection with a stimulation device that is configured or configurable to delivery cardiac therapy and/or sense information germane to cardiac therapy.

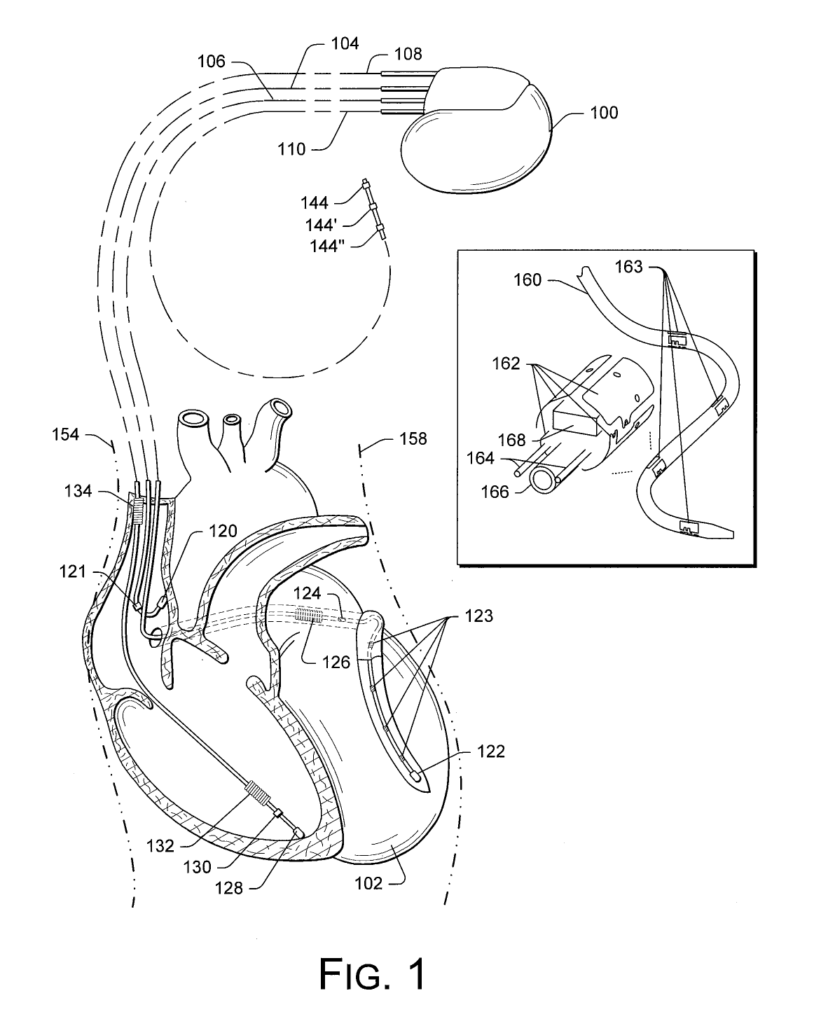

FIG. 1 shows an exemplary stimulation device 100 in electrical communication with a patient's heart 102 by way of three leads (a right atrial lead 104, a left ventricular lead 106 and a right ventricular lead 108), suitable for delivering multi-chamber stimulation and shock therapy. The leads 104, 106, 108 are optionally configurable for delivery of stimulation pulses suitable for stimulation of nerves or other tissue. In addition, in the example of FIG. 1, the device 100 includes a fourth lead 110 having multiple electrodes 144, 144′, 144″ suitable for stimulation of tissue and/or sensing of physiologic signals. This lead may be positioned in and/or near a patient's heart and/or remote from the heart.

FIG. 1 also shows approximate locations of the right and left phrenic nerves 154, 158. The phrenic nerve is made up mostly of motor nerve fibres for producing contractions of the diaphragm. In addition, it provides sensory innervation for various components of the mediastinum and pleura, as well as the upper abdomen (e.g., liver and gall bladder). The right phrenic nerve 154 passes over the brachiocephalic artery, posterior to the subclavian vein, and then crosses the root of the right lung anteriorly and then leaves the thorax by passing through the vena cava hiatus opening in the diaphragm at the level of T8. More specifically, with respect to the heart, the right phrenic nerve 154 passes over the right atrium while the left phrenic nerve 158 passes over the pericardium of the left ventricle and pierces the diaphragm separately. While certain therapies may call for phrenic nerve stimulation (e.g., for treatment of sleep apnea), in general, cardiac pacing therapies avoid phrenic nerve stimulation through judicious lead and electrode placement, selection of electrode configurations, adjustment of pacing parameters, etc.

Referring again to the various leads of the device 100, the right atrial lead 104, as the name implies, is positioned in and/or passes through a patient's right atrium. The right atrial lead 104 is configured to sense atrial cardiac signals and/or to provide right atrial chamber stimulation therapy. As described further below, the right atrial lead 104 may be used by the device 100 to acquire far-field ventricular signal data. As shown in FIG. 1, the right atrial lead 104 includes an atrial tip electrode 120, which typically is implanted in the patient's right atrial appendage, and an atrial ring electrode 121. The right atrial lead 104 may have electrodes other than the tip 120 and ring 121 electrodes. Further, the right atrial lead 104 may include electrodes suitable for stimulation and/or sensing located on a branch.

To sense atrial cardiac signals, ventricular cardiac signals and/or to provide chamber pacing therapy, particularly on the left side of a patient's heart, the stimulation device 100 is coupled to the left ventricular lead 106, which in FIG. 1 is also referred to as a coronary sinus lead as it is designed for placement in the coronary sinus and/or tributary veins of the coronary sinus. As shown in FIG. 1, the coronary sinus lead 106 is configured to position at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

In the example of FIG. 1, the coronary sinus lead 106 includes a series of electrodes 123. In particular, a series of four electrodes are shown positioned in an anterior vein of the heart 102. Other coronary sinus leads may include a different number of electrodes than the lead 106. As described herein, an exemplary method selects one or more electrodes (e.g., from electrodes 123 of the lead 106) and determines characteristics associated with conduction and/or timing in the heart to aid in ventricular pacing therapy and/or assessment of cardiac condition. As described in more detail below, an illustrative method acquires information using various electrode configurations where an electrode configuration typically includes at least one electrode of a coronary sinus lead or other type of left ventricular lead. Such information may be used to determine a suitable electrode configuration for the lead 106 (e.g., selection of one or more electrodes 123 of the lead 106).

In the example of FIG. 1, as connected to the device 100, the coronary sinus lead 106 is configured for acquisition of ventricular cardiac signals (and optionally atrial signals) and to deliver left ventricular pacing therapy using, for example, at least one of the electrodes 123 and/or the tip electrode 122. The lead 106 optionally allows for left atrial pacing therapy, for example, using at least the left atrial ring electrode 124. The lead 106 optionally allows for shocking therapy, for example, using at least the left atrial coil electrode 126. For a complete description of a particular coronary sinus lead, the reader is directed to U.S. Pat. No. 5,466,254, “Coronary Sinus Lead with Atrial Sensing Capability” (Helland), which is incorporated herein by reference.

The stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108, as connected to the device 100, is capable of sensing or receiving cardiac signals, and delivering stimulation in the form of pacing and shock therapy to the right ventricle. An exemplary right ventricular lead may also include at least one electrode capable of stimulating other tissue; such an electrode may be positioned on the lead or a bifurcation or leg of the lead. A right ventricular lead may include a series of electrodes, such as the series 123 of the left ventricular lead 106.

FIG. 1 also shows a lead 160 as including several electrode arrays 163. In the example of FIG. 1, each electrode array 163 of the lead 160 includes a series of electrodes 162 with an associated circuit 168. Conductors 164 provide an electrical supply and return for the circuit 168. The circuit 168 includes control logic sufficient to electrically connect the conductors 164 to one or more of the electrodes of the series 162. In the example of FIG. 1, the lead 160 includes a lumen 166 suitable for receipt of a guidewire to facilitate placement of the lead 160. As described herein, any of the leads 104, 106, 108 or 110 may include one or more electrode array, optionally configured as the electrode array 163 of the lead 160.

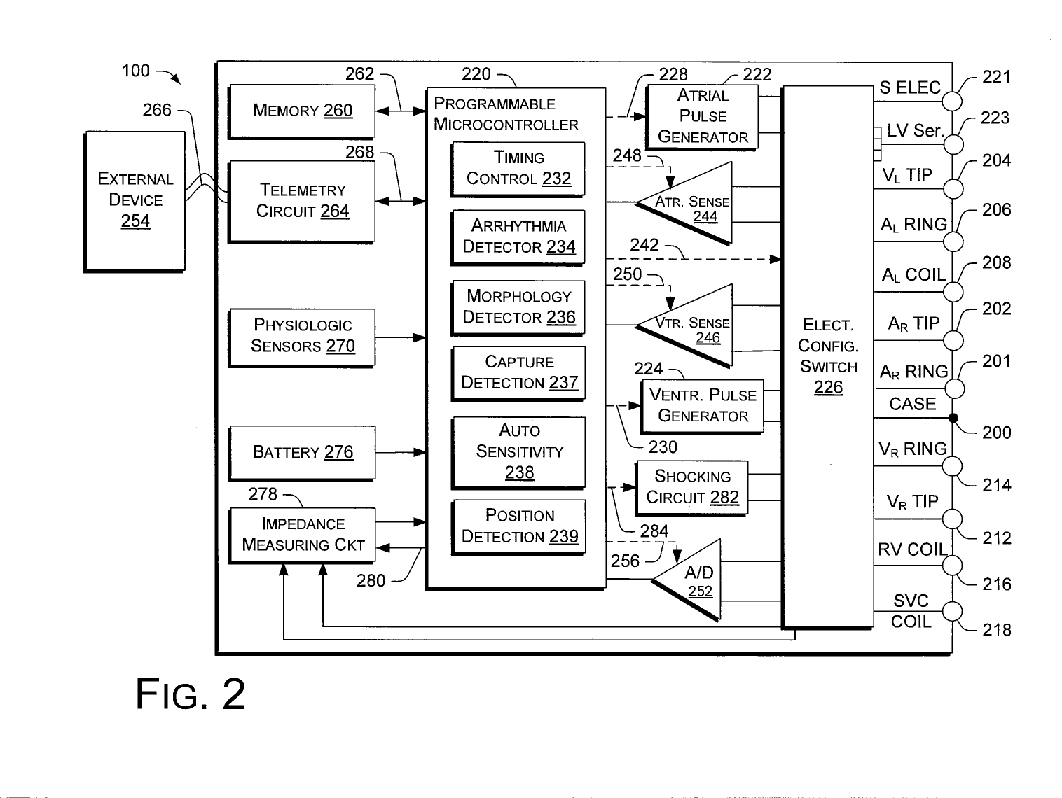

FIG. 2 shows an exemplary, simplified block diagram depicting various components of the device 100. The device 100 can be capable of treating both fast and slow arrhythmias with stimulation therapy, including cardioversion, defibrillation, and pacing stimulation. While a particular multi-chamber device is shown, it is to be appreciated and understood that this is for illustration purposes only. Thus, the techniques, methods, etc., described below can be implemented in connection with any suitably configured or configurable device. Accordingly, one of skill in the art could readily duplicate, eliminate, or disable the appropriate circuitry in any desired combination to provide a device capable of treating the appropriate chamber(s) or regions of a patient's heart.

Housing 200 for the device 100 is often referred to as the “can”, “case” or “case electrode”, and may be programmably selected to act as the return electrode for all “unipolar” modes. As described below, various exemplary techniques implement unipolar sensing for data that may include indicia of functional conduction block in myocardial tissue. Housing 200 may further be used as a return electrode alone or in combination with one or more of the coil electrodes 126, 132 and 134 for shocking or other purposes. Housing 200 further includes a connector (not shown) having a plurality of terminals 201, 202, 204, 206, 208, 212, 214, 216, 218, 221, 223 (shown schematically and, for convenience, the names of the electrodes to which they are connected are shown next to the terminals).

To achieve right atrial sensing, pacing and/or other tissue sensing, stimulation, etc., the connector includes at least a right atrial tip terminal (A_{R} TIP) 202 adapted for connection to the right atrial tip electrode 120. A right atrial ring terminal (A_{R} RING) 201 is also shown, which is adapted for connection to the right atrial ring electrode 121. To achieve left chamber sensing, pacing, shocking, and/or other tissue sensing, stimulation, etc., the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively. Connection to suitable stimulation electrodes is also possible via these and/or other terminals (e.g., via a stimulation terminal S ELEC 221). The terminal S ELEC 221 may optionally be used for sensing. For example, electrodes of the lead 110 may connect to the device 100 at the terminal 221 or optionally at one or more other terminals.

A terminal 223 allows for connection of a series of left ventricular electrodes. For example, the series of four electrodes 123 of the lead 106 may connect to the device 100 via the terminal 223. The terminal 223 and an electrode configuration switch 226 allow for selection of one or more of the series of electrodes and hence electrode configuration. In the example of FIG. 2, the terminal 223 includes four branches to the switch 226 where each branch corresponds to one of the four electrodes 123.

To support right chamber sensing, pacing, shocking, and/or other tissue sensing, stimulation, etc., the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively.

At the core of the stimulation device 100 is a programmable microcontroller 220 that controls the various modes of cardiac or other therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that is suitable to carry out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052, the state-machine of U.S. Pat. Nos. 4,712,555 and 4,944,298, all of which are incorporated by reference herein. For a more detailed description of the various timing intervals used within the stimulation device and their inter-relationship, see U.S. Pat. No. 4,788,980, also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

The microcontroller 220 further includes timing control circuitry 232 to control the timing of the stimulation pulses (e.g., pacing rate, atrio-ventricular (AV) delay, interatrial conduction (AA) delay, or interventricular conduction (VV) delay, etc.) as well as to keep track of the timing of refractory periods, blanking intervals, noise detection windows, evoked response windows, alert intervals, marker channel timing, etc., which is well known in the art.

The microcontroller 220 further includes an arrhythmia detector 234. The detector 234 can be utilized by the stimulation device 100 for determining desirable times to administer various therapies. The detector 234 may be implemented in hardware as part of the microcontroller 220, or as software/firmware instructions programmed into the device and executed on the microcontroller 220 during certain modes of operation.

Microcontroller 220 further includes a morphology discrimination module 236, a capture detection module 237 and an auto sensing module 238. These modules are optionally used to implement various exemplary recognition algorithms and/or methods presented below. The aforementioned components may be implemented in hardware as part of the microcontroller 220, or as software/firmware instructions programmed into the device and executed on the microcontroller 220 during certain modes of operation. The capture detection module 237, as described herein, may aid in acquisition, analysis, etc., of information relating to IEGMs and, in particular, act to distinguish capture versus non-capture versus fusion.

The microcontroller 220 further includes an optional position detection module 239. The module 239 may be used to acquire data germane to position of one or more electrodes. The module 239 may operate in conjunction with a device other than the device 100 (internal or external). The microcontroller 220 may initiate one or more algorithms of the module 239 in response to a signal detected by various circuitry or information received via the telemetry circuit 264. Such a module may help monitor cardiac mechanics in relationship to cardiac electrical activity and, in turn, may help to optimize cardiac resynchronization therapy based at least in part on such monitoring. The module 239 may operate in conjunction with various other modules and/or circuits of the device 100 (e.g., the impedance measuring circuit 278, the switch 226, the ND 252, etc.).

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial and ventricular sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each of the sensing circuits 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 may utilize the atrial and ventricular sensing circuits, 244 and 246, to sense cardiac signals to determine whether a rhythm is physiologic or pathologic. Of course, other sensing circuits may be available depending on need and/or desire. In reference to arrhythmias, as used herein, “sensing” is reserved for the noting of an electrical signal or obtaining data (information), and “detection” is the processing (analysis) of these sensed signals and noting the presence of an arrhythmia or of a precursor or other factor that may indicate a risk of or likelihood of an imminent onset of an arrhythmia.

The exemplary detector module 234, optionally uses timing intervals between sensed events (e.g., P-waves, R-waves, and depolarization signals associated with fibrillation) and to perform one or more comparisons to a predefined rate zone limit (i.e., bradycardia, normal, low rate VT, high rate VT, and fibrillation rate zones) and/or various other characteristics (e.g., sudden onset, stability, physiologic sensors, and morphology, etc.) in order to determine the type of remedial therapy (e.g., anti-arrhythmia, etc.) that is desired or needed (e.g., bradycardia pacing, anti-tachycardia pacing, cardioversion shocks or defibrillation shocks, collectively referred to as “tiered therapy”). Similar rules can be applied to the atrial channel to determine if there is an atrial tachyarrhythmia or atrial fibrillation with appropriate classification and intervention.

Cardiac signals are also applied to inputs of an analog-to-digital (ND) data acquisition system 252. The data acquisition system 252 is configured to acquire intracardiac electrogram (IEGM) signals or other action potential signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or another lead (e.g., the lead 110) through the switch 226 to sample cardiac signals or other signals across any pair or other number of desired electrodes. A control signal 256 from the microcontroller 220 may instruct the ND 252 to operate in a particular mode (e.g., resolution, amplification, etc.).

Various exemplary mechanisms for signal acquisition are described herein that optionally include use of one or more analog-to-digital converter. Various exemplary mechanisms allow for adjustment of one or more parameter associated with signal acquisition.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming and operation of the device 100.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms (IEGM) and other information (e.g., status information relating to the operation of the device 100, etc., as contained in the microcontroller 220 or memory 260) to be sent to the external device 254 through an established communication link 266.

The stimulation device 100 can further include one or more physiologic sensors 270. For example, the device 100 may include a “rate-responsive” sensor that may provide, for example, information to aid in adjustment of pacing stimulation rate according to the exercise state of the patient. However, the one or more physiological sensors 270 may further be used to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 responds by adjusting the various pacing parameters (such as rate, AV Delay, VV Delay, etc.) at which the atrial and ventricular pulse generators, 222 and 224, generate stimulation pulses.

While shown as being included within the stimulation device 100, it is to be understood that one or more of the physiologic sensors 270 may also be external to the stimulation device 100, yet still be implanted within or carried by the patient. Examples of physiologic sensors that may be implemented in device 100 include known sensors that, for example, sense respiration rate, oxygen concentration of blood, pH of blood, CO₂ concentration of blood, ventricular gradient, cardiac output, preload, afterload, contractility, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 which is hereby incorporated by reference.

The one or more physiologic sensors 270 optionally include sensors for detecting movement and minute ventilation in the patient. Signals generated by a position sensor, a MV sensor, etc., may be passed to the microcontroller 220 for analysis in determining whether to adjust the pacing rate, etc. The microcontroller 220 may monitor the signals for indications of the patient's position and activity status, such as whether the patient is climbing upstairs or descending downstairs or whether the patient is sitting up after lying down.

The stimulation device 100 additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 200 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to 0.5 J), moderate (e.g., 0.5 J to 10 J), or high energy (e.g., 11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (e.g., corresponding to thresholds in the range of approximately 5 J to 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

As already mentioned, the implantable device 100 includes impedance measurement circuitry 278. Such a circuit may measure impedance or electrical resistance through use of various techniques. For example, the device 100 may deliver a low voltage (e.g., about 10 mV to about 20 mV) of alternating current between the RV tip electrode 128 and the case electrode 200. During delivery of this energy, the device 100 may measure resistance between these two electrodes where the resistance depends on any of a variety of factors. For example, the resistance may vary inversely with respect to volume of blood along the path.

In another example, resistance measurement occurs through use of a four terminal or electrode technique. For example, the exemplary device 100 may deliver an alternating current between one of the RV tip electrode 128 and the case electrode 200. During delivery, the device 100 may measure a potential between the RA ring electrode 121 and the RV ring electrode 130 where the potential is proportional to the resistance between the selected potential measurement electrodes.

With respect to two terminal or electrode techniques, where two electrodes are used to introduce current and the same two electrodes are used to measure potential, parasitic electrode-electrolyte impedances can introduce noise, especially at low current frequencies; thus, a greater number of terminals or electrodes may be used. For example, aforementioned four electrode techniques, where one electrode pair introduces current and another electrode pair measures potential, can cancel noise due to electrode-electrolyte interface impedance. Alternatively, where suitable or desirable, a two terminal or electrode technique may use larger electrode areas (e.g., even exceeding about 1 cm²) and/or higher current frequencies (e.g., above about 10 kHz) to reduce noise.

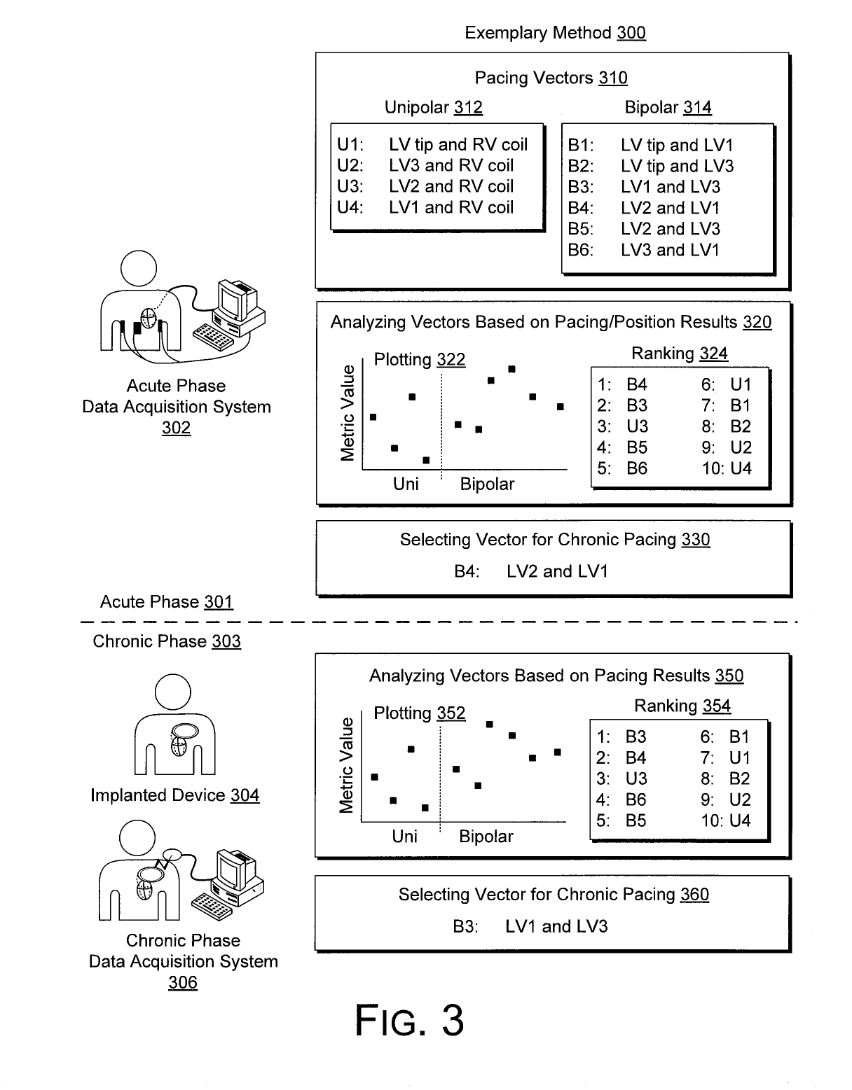

FIG. 3 shows an exemplary method 300 with respect to an acute phase 301 and a chronic phase 303 along with equipment 302, 304 and 306 that may be used to implement the method 300. In the example of FIG. 3, the method acts to select an optimal configuration in the acute phase 301 and to allow for selection of an optimal configuration in the chronic phase 303, per an optimization algorithm implemented in the chronic phase 303. In accordance with the invention described herein, such an optimization algorithm may be validated in an acute phase, for example, as more information is typically available in an acute phase. Noting, however, that time and resources can become limiting factors in an acute or intraoperative phase.

In the acute phase 301, the method 300 commences in a pacing vectors block 310 that, for the example of FIG. 3, lists four unipolar vectors 312 and six bipolar vectors 314. An analysis block 320 includes ranking vectors based on pacing results, position results or a combination of pacing and position results. For example, a plotting block 322 includes a plot of values for a metric for the four unipolar vectors and the six bipolar vectors and a ranking block 324 includes a ranking of the ten vectors based on the values for the metric. As described further below, metrics may include vector magnitude as a function of time, vector area as a function of time, electrode volume as a function of time, mechanical dyssynchrony, electrical activation and mechanical activation delay, A selection block 330 includes selecting a vector for chronic pacing based on the analysis. For example, as the vector B4 was ranked first, as having the highest metric value, the method 300 selects this vector for chronic pacing in the chronic phase 303.

In the chronic phase 303, the method 300 may continue according to a schedule, occurrence of an event, an in-clinic follow-up visit, etc. (see, e.g., various pending U.S. patent applications (Ser. Nos. 12/621,373; 12/398,460; 12/639,788; and 12/553,413); and U.S. Patent Application Publication Nos. 2009/0318995 and 2009/0306732; as cited in the description below). The method 300 may maintain the vector selected in the acute phase 301 or it may select a different vector for chronic pacing.

In the chronic phase 303, per an analysis block 350, the method 300 includes analyzing electrode configuration vectors based on pacing results. In the example of FIG. 3, a plotting block 352 includes a plot of values for a metric for the four unipolar vectors and the six bipolar vectors and a ranking block 354 includes a ranking of the ten vectors based on the values for the metric. Exemplary metrics in the chronic phase include AV delay, VV delay, ER/capture threshold and cardiogenic impedance. A selection block 360 includes selecting a vector for chronic pacing based on the analysis. For example, as the vector B3 was ranked first, as having the highest metric value, the method 300 selects this vector for chronic pacing in the chronic phase 303.

As indicated in the example of FIG. 3, the equipment available to acquire information during the acute phase 301 usually provides more data acquisition options and typically more accurate and meaningful measurements than the equipment available to acquire information during the chronic phase 303. The acute phase data acquisition system 302 includes a computing device, catheters and surface electrodes. As described herein, the system 302 may be a localization system such as the ENSITE® NAVX® localization system (St Jude Medical, Atrial Fibrillation Division). In contrast, the chronic phase 303 relies on an implanted device 304 and, optionally, a chronic phase data acquisition system 306, such as the A/D converter 252 and the microcontroller 220 previously described with reference to FIG. 2. As described herein, the system 306 may be an implantable device programmer such as the 3510 programmer or the MERLIN® programmer (St. Jude Medical CRMD).

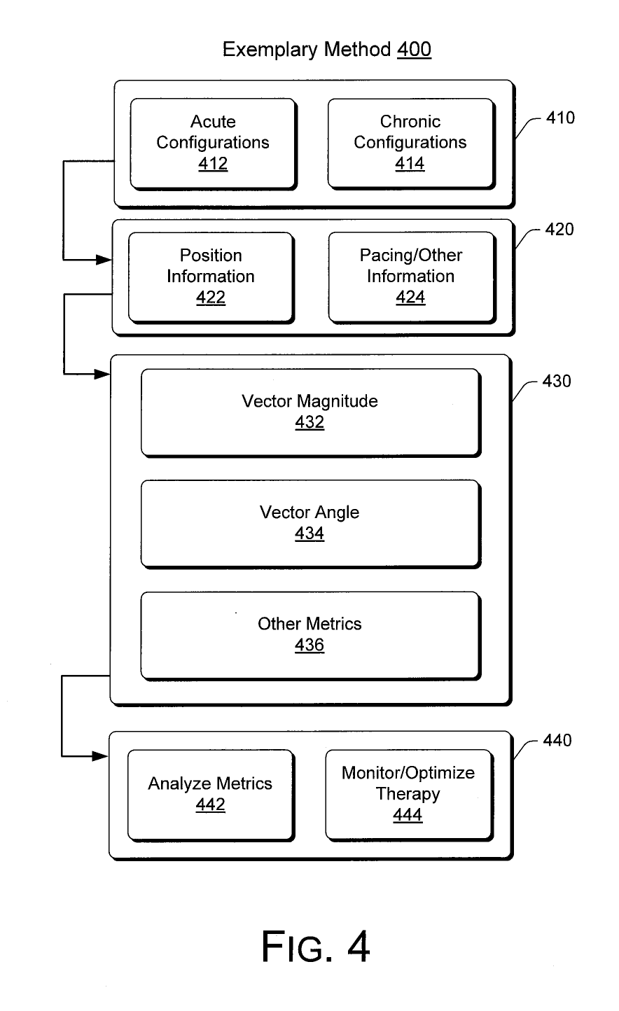

FIG. 4 shows an exemplary method 400 for acquiring position information and calculating one or more metrics 430. In the example of FIG. 4, the method 400 includes a configurations block 410 that includes acute or intraoperative configurations 412 and chronic configurations 414. The acute configurations 412 pertain to configurations that may be achieved during an operative procedure. For example, during an operative procedure, one or more leads (and/or catheter(s)) may be positioned in a patient where the one or more leads are connected to, or variously connectable to, a device configured to acquire information, such as electrical and position information, and optionally to deliver electrical energy to the patient (e.g., to the heart, to a nerve, to other tissue, etc.). The chronic configurations 414 pertain to configurations achievable by a chronically implanted device and its associated lead or leads. In general, acute configurations include those achievable by physically re-positioning a lead (or catheter) in a patient's body while chronic configurations normally do not allow for re-positioning as a lead or leads are usually anchored during implantation or become anchored in the weeks to months after implantation. Chronic configurations do, however, include selection of a subset of the multiple implanted electrodes, for example using the tip electrode versus the first ring electrode as a cathode or using the tip and first ring as a bipolar pair versus using the tip and ring as two independent cathodes. Thus, acute configurations include configurations available by changing device settings, electrode selection, and physical position of electrodes, while chronic configurations include only those configurations available by changing device settings and electrode selection, or “electronic repositioning” of one or more stimulation electrodes.

As indicated in FIG. 4, an acquisition block 420 includes acquisition of position information 422 and optionally acquisition of pacing and/or other information 424 (e.g., electrical information as to electrical activity of the heart, biosensor information, etc.). While an arrow indicates that a relationship or relationships may exist between the configurations block 410 and the acquisition block 420, acquisition of information may occur by using in part an electrode (or other equipment) that is not part of a configuration. For example, the acquisition block 420 may rely on one or more surface electrodes that define a coordinate system or location system for locating an electrode that defines one or more configurations. For example, three pairs of surface electrodes positioned on a patient may be configured to deliver current and define a three-dimensional space whereby measurement of a potential locates an electrode in the three-dimensional space.

As described herein, an electrode may be configured for delivery of energy to the body; for acquisition of electrical information; for acquisition of position information; for acquisition of electrical information and position information; for delivery of energy to the body and for acquisition of electrical information; for delivery of energy to the body and for acquisition of position information; for delivery of energy to the body, for acquisition of electrical information and for acquisition of position information.

In various examples, acquisition of position information occurs by measuring one or more potentials where the measuring relies on an electrode that assists in determining a position of the electrode or other item (e.g., a lead or sensor) where the electrode may also be configured to sense signals and/or deliver energy to the body (e.g., electrical energy to pace a chamber of the heart). For example, an electrode may deliver energy sufficient to stimulate the heart and then be tracked along one or more dimensions to monitor the position information resulting from the stimulation. Further, such an electrode may be used to acquire electrical information (e.g., an IEGM that evidences an evoked response). Such an electrode can perform all three of these tasks with proper circuitry and control. For example, after delivery of the energy, the electrode may be configured for acquiring one or more potentials related to position and for acquiring an electrogram. To acquire potentials and an electrogram, circuitry may include gating or other sampling techniques (e.g., to avoid circuitry or interference issues). Such circuitry may rely on one sampling frequency for acquiring potentials for motion tracking and another sampling frequency for acquiring an electrogram.

In the method 400 of FIG. 4, a metrics block 430 includes vector magnitude 432, vector angle 434 and other metrics 436. As described herein, vector metrics are based, at least in part, on acquired position information. As described herein, a vector may be defined between two electrodes and may be referenced with respect to one or more other electrodes. For example, a triangle may be defined between a right atrial electrode, a right ventricular electrode and an electrode in a vein of a lateral wall of the left ventricle. An analysis of position of these electrodes with respect to time may indicate that two of the electrodes exhibit less movement over a cardiac cycle when compared to a third electrode. In such a scenario, the two electrodes that exhibit less movement may be used as a reference or references to more accurately track a vector with its vector tip defined by the position of third electrode. As described herein, trials demonstrate that an electrode located in the apex of the right ventricle moves less than an electrode located along the lateral wall of the left ventricle (e.g., in a tributary vein of the coronary sinus). In trials, various aspects of a vector defined by an electrode located in right ventricular apex to an electrode located in a vein of the lateral wall of the left ventricle were analyzed with respect to indicators of cardiac performance. The results demonstrate that such an RV-to-LV vector can be used as an indicator of cardiac performance.

In the example of FIG. 4, the conclusion block 440 may perform actions such as to analyze metrics 442 and/or to optimize or monitor patient and/or device condition 444 based on one or more of the metrics 430.

With respect to configurations of the configuration block 410, an exemplary method may rely on various configurations (C1, C2, . . . Cn) that may be combined with intervention options. As mentioned, a configuration may be defined based on factors such as electrode location (e.g., with respect to some physiological feature of the heart or another electrode), stimulation parameters for an electrode or electrodes and, where appropriate, one or more interelectrode timings. Hence, with reference to FIG. 1, C1 may be a configuration that relies on the RV tip electrode 128, the RV ring electrode 130, the LV tip electrode 122 and the LV ring electrode 124 while C2 may be a configuration that relies on the same electrodes as C1 but where the stimulation polarity for the LV electrodes is reversed. Further, C3 may rely on the same electrodes where the timing between delivery of a stimulus to the RV and delivery of a stimulus to the LV is different compared to C1. Yet further, C4 may rely on the same electrodes where the duration of a stimulus to the RV is different compared to C1. In these foregoing examples, configurations provide for one or more electrodes to deliver energy to stimulate the right ventricle and for one or more electrodes to deliver energy to stimulate the left ventricle. In other examples, configurations may provide for stimulation of a single chamber at one or more sites, stimulation of one chamber at a single site and another chamber at multiple sites, multiple chambers at multiple sites per chamber, etc.

As mentioned, configurations can include one or more so-called “stimulators” and/or “sensors”. Thus, the configurations block 410 may select a configuration that includes one or more of an electrode, a lead, a catheter, a device, etc. In various examples, a stimulator or a sensor can include one or more electrodes configured to measure a potential or potentials to thereby directly or indirectly provide position information for the stimulator or the sensor. For example, a lead-based oximeter (oxygen sensor) may include an electrode configured to measure a potential for providing position information for the oximeter or a lead-based RF applicator may include electrodes configured to measure potentials for providing position information for the RF applicator or a tip of the lead.

As described herein, an exemplary acute phase method can include: locating one or more electrodes within the heart and/or surrounding space (e.g., intra-chamber, intra-vascular, intrapericardial, etc., which may be collectively referred to as “cardiac space”); and acquiring information (e.g., via one or more measured potentials) to calculate one or more metrics for at least one of the one or more electrodes using an electroanatomic mapping system (e.g., the ENSITE® NAVX® system or other system with appropriate features). In such a method, the located electrodes may be configured for acquisition of electrical information indicative of physiological function (e.g., IEGMs, muscle signals, nerve signals, etc.). Further, with respect to acquisition of information, an acquisition system may operate at an appropriate sampling rate. For example, an acquisition system for position information may operate at a sampling rate of about 100 Hz (e.g., the ENSITE® NAVX® system can sample at about 93 Hz) and an acquisition system for electrical information may operate at a sampling rate of about 1200 Hz (e.g., in unipolar, bipolar or other polar arrangement).

An exemplary acute phase method may include preparing a patient for both implant of a device such as the device 100 of FIGS. 1 and 2 and for electroanatomic mapping study. Such preparation may occur in a relatively standard manner for implant prep, and using the ENSITE® NAVX® system or other similar technology for the mapping prep. As described herein, any of a variety of electroanatomic mapping or locating systems that can locate indwelling electrodes in and around the heart may be used.

Once prepped, a clinician may place leads and/or catheters in the patient's body, including any leads to be chronically implanted as part of a therapy system (e.g., CRT), as well as optional additional electrodes that may yield additional information (e.g., to increase accuracy by providing global information or other information).

After an initial placement of an electrode-bearing catheter or an electrode-bearing lead, a clinician may then connect one or more electrodes to an electroanatomic mapping or localizing system. The term “connection” can refer to physical electrical connection or wireless connection (e.g., telemetric, RF, ultrasound, etc.) with the electrodes or wireless connection with another device that is in electrical contact with the electrodes.

Once an appropriate connection or connections have been made, real-time position data for one or more electrodes may be acquired for various configurations or conditions. For example, position data may be acquired during normal sinus rhythm; pacing in one or more chambers; advancing, withdrawing, or moving a location of an electrode; pacing one or more different electrode configurations (e.g. multisite pacing); or varying inter-stimulus timing (e.g. AV delay, VV delay).

In various examples, simultaneous to the position recording, an intracardiac electrogram (IEGM) from each electrode can also be recorded and associated with the anatomic position of the electrode. While various examples refer to simultaneous acquisition, acquisition of electrical information and acquisition of position information may occur sequentially (e.g., alternate cardiac cycles) or interleaved (e.g., both acquired during the same cardiac cycle but offset by sampling time or sampling frequency).

In various exemplary acute phase methods, electrodes within the cardiac space may be optionally positioned at various locations (e.g., by continuous movement or by discrete, sequential moves), with a mapping system recording the real-time position information at each electrode position in a point-by-point manner. Such position data can by associated with a respective anatomic point from which it was collected. By moving the electrodes from point to point during an intervention, the position data from each location can be incorporated into a single map, model, or parameter.

As explained, an exemplary acute phase method may include mapping one or more metrics. In turn, an algorithm (e.g., with clinician assistance) may select a configuration (e.g., electrode location, multisite arrangement, AV/VV timing) that yielded the best value for a metric and use the selected configuration as a chronic configuration for the CRT system. Such a chronic configuration may be optionally updated from time to time (e.g., during a follow-up visit, in a patient environment, etc., depending on specific capabilities of a system).

Various exemplary acute phase methods, using either a single metric or a combination of more than one metric, may automatically select a configuration, present an optimal configuration for acknowledgement by a clinician, or present various configurations to a clinician.

As discussed herein, various exemplary techniques deliver current and measure potential where potential varies typically with respect to cardiac mechanics (e.g., due to motion). For example, electrodes for delivery of current may be placed at locations that do not vary significantly with respect to cardiac mechanics or other patient motion (e.g., breathing) while one or more electrodes for measuring potential may be placed at a location or locations that vary with respect to cardiac mechanics or other patient motion. Alternatively, electrodes for measuring potential may be placed at locations that do not vary significantly with respect to cardiac mechanics or other patient motion while one or more electrodes for delivery of current may be placed at a location or locations that vary with respect to cardiac mechanics or other patient motion. Various combinations of the foregoing arrangements are possible as well. Electrodes may be associated with a catheter or a lead. In some instances, an electrode may be a “stand-alone” electrode, such as a case electrode of an implantable device (see, e.g., the case electrode 200 of the device 100 of FIGS. 1 and 2).

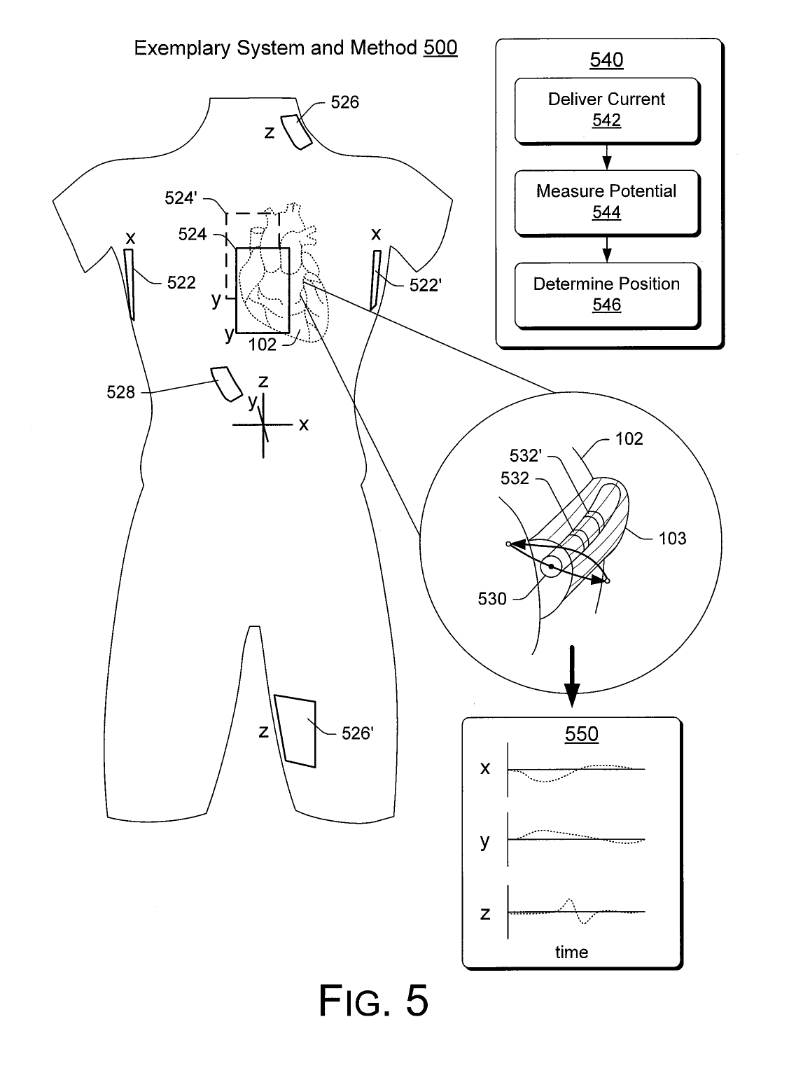

FIG. 5 shows an arrangement and method 500 that may rely in part on a commercially available system marketed as the ENSITE® NAVX® navigation and visualization system (see also LocaLisa system). The ENSITE® NAVX® system is a computerized storage and display system for use in electrophysiology studies of the human heart. The system consists of a console workstation, patient interface unit, and an electrophysiology mapping catheter and/or surface electrode kit. By visualizing the global activation pattern seen on color-coded isopotential maps in the system, in conjunction with the reconstructed electrograms, an electrophysiologist can identify the source of an arrhythmia and can navigate to a defined area for therapy. The ENSITE® system is also useful in treating patients with simpler arrhythmias by providing non-fluoroscopic navigation and visualization of conventional electrophysiology (EP) catheters.

As shown in FIG. 5, electrodes 532, 532′, which may be part of a standard EP catheter 530 (or lead), sense electrical potential associated with current signals transmitted between three pairs of surface electrode patches 522, 522′ x-axis), 524, 524′ (y-axis) and 526, 526′ (z-axis). An addition electrode patch 528 is available for reference, grounding or other function. The ENSITE® NAVX® System can also collect electrical data from a catheter and can plot a cardiac electrogram from a particular location (e.g., cardiac vein 103 of heart 102). Information acquired may be displayed as a 3-D isopotential map and as virtual electrograms. Repositioning of the catheter allows for plotting of cardiac electrograms from other locations. Multiple catheters may be used as well. A cardiac electrogram or electrocardiogram (ECG) of normal heart activity (e.g., polarization, depolarization, etc.) typically shows atrial depolarization as a “P wave”, ventricular depolarization as an “R wave”, or QRS complex, and repolarization as a “T wave”. The ENSITE® NAVX® system may use electrical information to track or navigate movement and construct three-dimensional (3-D) models of a chamber of the heart.

A clinician can use the ENSITE® NAVX® system to create a 3-D model of a chamber in the heart for purposes of treating arrhythmia (e.g., treatment via tissue ablation). To create the 3-D model, the clinician applies surface patches to the body. The ENSITE® NAVX® system transmits an electrical signal between the patches and the system then senses the electrical signal using one or more catheters positioned in the body. The clinician may sweep a catheter with electrodes across a chamber of the heart to outline structure. Signals acquired during the sweep, associated with various positions, can then be used to generate a 3-D model. A display can display a diagram of heart morphology, which, in turn, may help guide an ablation catheter to a point for tissue ablation.

With respect to the foregoing discussion of current delivery and potential measurement, per a method 540, a system (e.g., such as the ENSITE® NAVX® system) delivers low level separable currents from the three substantially orthogonal electrode pairs (522, 522′, 524, 524′, 526, 526′) positioned on the body surface (delivery block 542). The specific position of a catheter (or lead) electrode within a chamber of the heart can then be established based on three resulting potentials measured between the recording electrode with respect to a reference electrode, as seen over the distance from each patch set to the recording tip electrode (measurement block 544). Sequential positioning of a catheter (or lead) at multiple sites along the endocardial surface of a specific chamber can establish that chamber's geometry, i.e., position mapping (position/motion determination block 546). Where the catheter (or lead) 530 moves, the method 540 may also measure motion.

In addition to mapping at specific points, the ENSITE® NAVX® system provides for interpolation (mapping a smooth surface) onto which activation voltages and times can be registered. Around 50 points are required to establish a surface geometry and activation of a chamber at an appropriate resolution. The ENSITE® NAVX® system also permits the simultaneous display of multiple catheter electrode sites, and also reflects real-time motion of both ablation catheters and those positioned elsewhere in the heart.

The ENSITE® NAVX® system relies on catheters for temporary placement in the body. Various exemplary techniques described herein optionally use one or more electrodes for chronic implantation. Such electrodes may be associated with a lead, an implantable device, or other chronically implantable component. Referring again to FIG. 4, the configuration block 410 indicates that acute or intraoperative configurations 412 and chronic configurations 414 may be available. Acute configurations 412 may rely on a catheter and/or a lead suitable for chronic implantation.

With respect to motion (e.g., change in position with respect to time), the exemplary system and method 500 may track motion of an electrode in one or more dimensions. For example, a plot 550 of motion versus time for three dimensions corresponds to motion of one or more electrodes of the catheter (or lead) 530 positioned in a vessel 103 of the heart 102 where the catheter (or lead) 530 includes the one or more electrodes 532, 532′. Two arrows indicate possible motion of the catheter (or lead) 530 where hysteresis may occur over a cardiac cycle. For example, a systolic path may differ from a diastolic path.

The exemplary method 540, as mentioned, includes the delivery block 542 for delivery of current, the measurement block 544 to measure potential in a field defined by the delivered current and the determination block 546 to determine position or motion based at least in part on the measured potential.

The system 500 may use one or more features of the aforementioned ENSITE® NAVX® system. For example, one or more pairs of electrodes (522, 522′, 524, 524′, 526, 526′ and optionally 528) may be used to define one or more dimensions by delivering an electrical signal or signals to a body and/or by sensing an electrical signal or signals. Such electrodes (e.g., patch electrodes) may be used in conjunction with one or more electrodes positioned in the body (e.g., the electrodes 532, 532′).

The exemplary system 500 may be used to track position or motion of one or more electrodes due to systolic function, diastolic function, respiratory function, etc. Electrodes may be positioned along the endocardium and/or epicardium during a scouting or mapping process for use in conjunction with electrical information.

With respect to stimulation, stimulation may be delivered to control cardiac mechanics (e.g., contraction of a chamber of the heart) and position or motion information may be acquired where such information is associated with the controlled cardiac mechanics. An exemplary selection process may identify the best stimulation site based on factors such as electrical activity, electromechanical delay, extent of motion, synchronicity of motion where motion may be classified as motion due to systolic function or motion due to diastolic function. In general, motion information corresponds to motion of an electrode or electrodes (e.g., endocardial electrodes, epicardial electrodes, etc.) and may be related to motion of the heart or other physiology.

As described with respect to FIG. 5, a localization system can acquire position information for one or more electrodes on a lead or catheter. The ENSITE® NAVX® system can operate at a sampling frequency around 100 Hz (10 ms), which, for a cardiac rhythm of 60 bpm, allows for 100 samples per electrode per cardiac cycle. In various examples, sampling may be gated to occur over only a portion of a cardiac cycle. Gating may rely on fiducial markers such as peaks, gradients, crossings, etc., in an electrogram of heart activity. Other techniques for gating can include accelerometer techniques, impedance techniques, pressure techniques, flow techniques, etc. For example, an accelerometer signal slope above a threshold value (e.g., due to cardiac contraction or relaxation) can be used to commence acquisition of information or to terminate acquisition of information during a cardiac cycle. Such a technique may be repeated over multiple cardiac.

As described herein, for one or more electrodes, a localization system provides four-dimensional information (e.g., x, y, z and time). The four-dimensional information describes a three-dimensional trajectory in space that can be analyzed or displayed in part, in whole or at one or more key points in time. As mentioned, various other types of information may be used to gate acquisition or to delineate points or segments of a trajectory.

As mentioned with respect to FIG. 3, an exemplary method may rely on defined vectors or relationships between electrodes. For example, unipolar and bipolar configurations are defined by relationships between electrodes. A so-called “pacing vector” refers to a vector defined by active electrodes relied on for delivery of pacing energy (e.g., anode/cathode). As described herein, a vector may be defined by active electrodes, passive electrodes or a combination of passive and active electrodes. A passive electrode is an electrode that may be used to sense information such as position information or IEGM or one or more other biosignals.

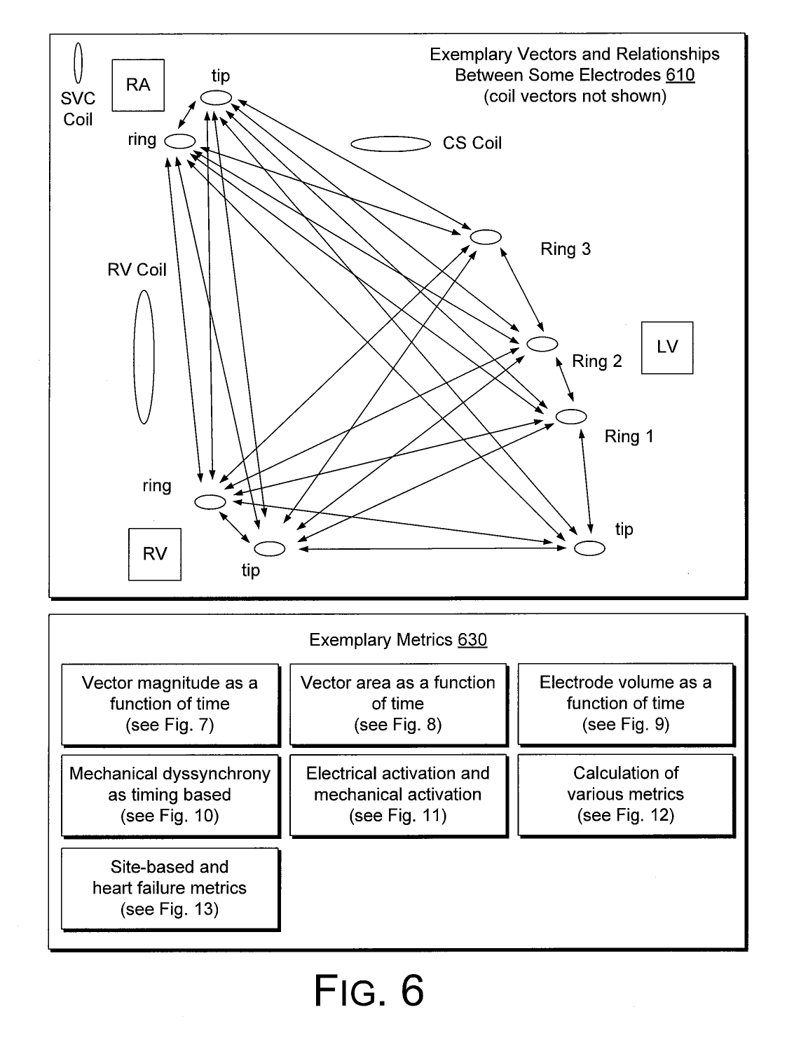

FIG. 6 shows a diagram of exemplary vectors and relationships between electrodes 610 along with various exemplary metrics 630. The exemplary vectors and relationships 610 pertain to two electrodes positioned in the right atrium, two electrodes positioned in the right ventricle and four electrodes positioned along a lateral wall of the left ventricle. Given these eight electrodes, twenty-five vectors may be defined as well as twenty-five one-to-one relationships (e.g., electrode-to-electrode). With the addition of a right ventricular coil electrode and a coronary sinus coil electrode, even more vectors and relationships are possible.

The electrodes in FIG. 6 may correspond to various electrodes in FIG. 1. For example, the right atrial electrodes may correspond to those of the RA lead 104, the left ventricular electrodes may correspond to those of the LV lead 106 and the right ventricular electrodes may correspond to those of the RV lead 108. Further, one or more of the electrodes in FIG. 6 may be multi-electrodes such as the multi-electrodes 163. Accordingly, the number of vectors and relationships may be increased significantly where one or more multi-electrodes are used (e.g., the number of vectors and relationships may exceed one hundred). Where the number of vectors and relationships is large, various exemplary techniques may be employed to expedite processing (e.g., selection, analysis, optimization, etc.).

According to the example of FIG. 6, by using electrodes from three leads (e.g., RV, LV, and RA leads), the ENSITE® NAX® system or other localization system can map motion and monitor distance and angular changes for a number of vector configurations during a cardiac cycle. As mentioned, an RV coil can also be utilized to create additional vectors, for example, to demonstrate wall-to-wall or region-to-region motion of the high to mid-RV septal region to a different region (e.g., a region in the LV, in the RV apex, or in the RA).

In the example of FIG. 6, the exemplary metrics 630 include vector magnitude as a function of time, vector area as a function of time, electrode volume as a function of time, mechanical dyssynchrony as being based on timing of stimulation or stimuli (e.g., energy delivery to the heart), electrical activation and mechanical activation, and heart failure. Such metrics may optionally be cast as part of an optimization problem.

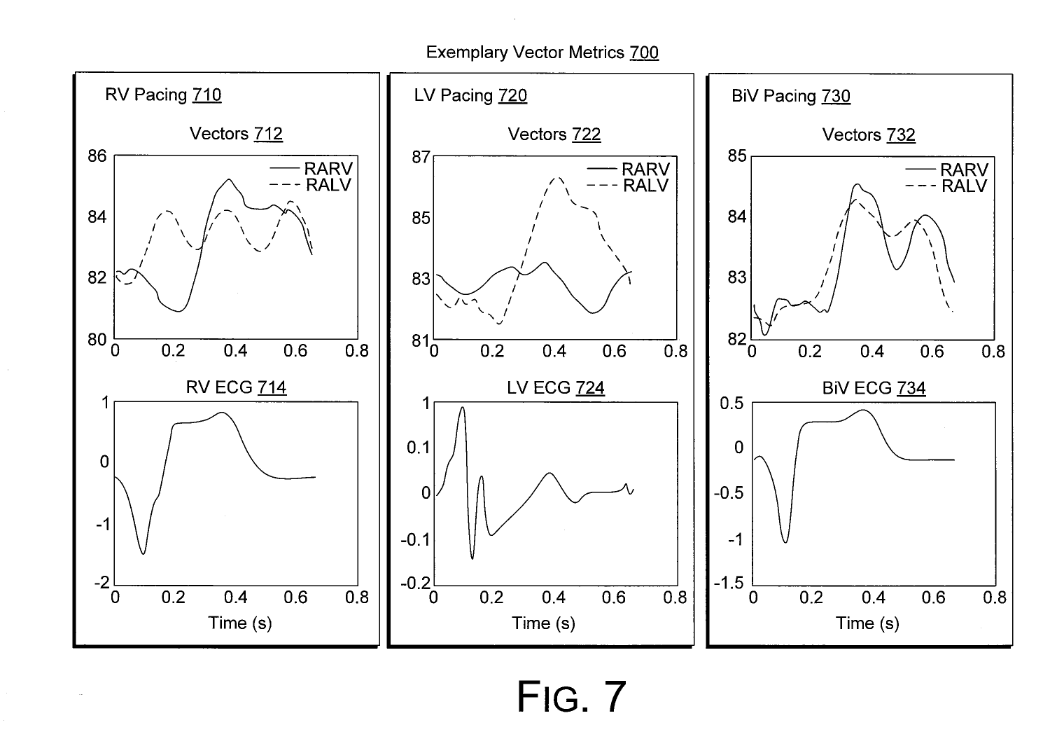

With respect to vector magnitude as a function of time, FIG. 7 shows various vector metric plots 700 for an RV pacing scenario 710, a LV pacing scenario 720 and a biventricular (BiV) pacing scenario 730. Each scenario includes a plot of RA-to-RV vector magnitude and RA-to-LV vector magnitude versus time 712, 722 and 732 and a corresponding ECG plot 714, 724 and 734.

The vector metric plots 712, 722 and 732 demonstrate how the heart moves responsive to RV pacing 710, LV pacing 720 and biventricular pacing 730 scenarios. The plot 712 shows that, for the RV pacing scenario 710, the displacement of the RA-to-RV vector is greater than that of the RA-to-LV vector. The plot 722 shows that, for the LV pacing scenario 720, the displacement of the RA-to-RV vector is less than that of the RA-to-LV vector. The plot 732 shows that, for the biventricular pacing scenario 730, the displacement of the RA-to-RV vector is about the same as that of the RA-to-LV vector.

In FIG. 7, the vector magnitude versus time plots may be referred to as vector magnitude waveforms where each of the waveforms corresponds to a cardiac cycle (or part of a cardiac cycle). These waveforms can be acquired for different conditions (e.g., pacing configurations, patient activity, etc.) and analyzed for features such as systolic slope, diastolic slope, peak-to-peak magnitude, local peak-to-peak, number of peaks, morphological analysis, baseline distance, longitudinal/circumferential fractional shortening, etc. In addition, one or more angular components from the vectors can be analyzed to compute metrics such as ventricular torsion and rotational direction or force.

Further details on vector-magnitude based metrics are provided in U.S. patent application Ser. No. 12/621,373 (assigned in its entirety to Pacesetter, Inc.), titled “Cardiac Resynchronization Therapy Optimization Using Vector Measurements Obtained from Realtime Electrode Position Tracking,” the disclosure of which is hereby incorporated by reference.

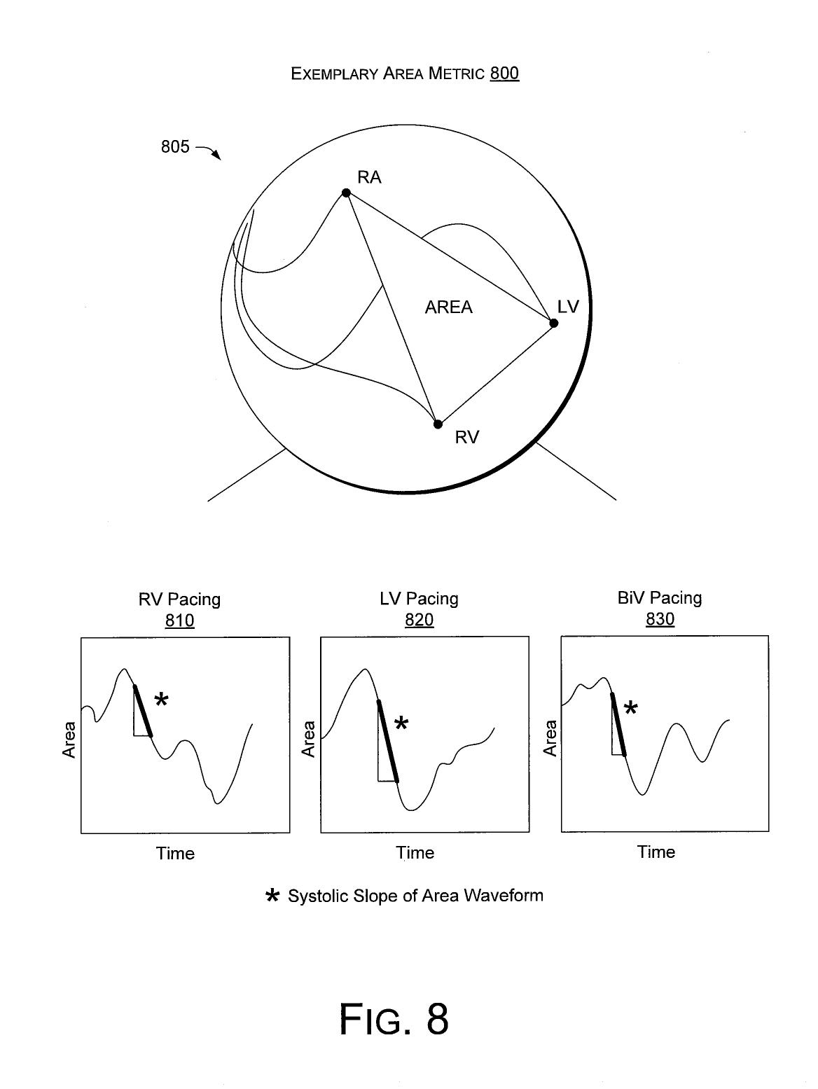

FIG. 8 shows an exemplary area metric 800 with reference to a radiograph 805 and a plot 810 for an RV pacing scenario, a plot 820 for a LV pacing scenario and a plot 830 for a biventricular pacing scenario (BiV). As described herein, area defined by three or more electrodes can be an indicator of cardiac contractility. For example, an area can be defined by connecting three or more electrodes during a cardiac cycle and such an area can be monitored with respect to time for different pacing interventions. An exemplary method can acquire position information to determine an area (e.g., as defined between selected electrodes). Such information may be acquired during at least a portion of a cardiac cycle and referred to as an area waveform, as shown in the plots 810, 820 and 830. Such a method can extract features from such waveforms where such features may include systolic slope, diastolic slope, peak-to-peak magnitude, local peak-to-peak, number of peaks, morphological analysis, baseline area, longitudinal/circumferential fractional shortening, etc. As described herein, exemplary features include angular changes of an area plane during a cardiac cycle, which can be analyzed to compute metrics such as ventricular torsion and rotational direction or force. In the example of FIG. 8, the plots 810, 820 and 830 include systolic slope, indicated by an asterisk.

Further details on area based metrics are provided in U.S. patent application Ser. No. 12/398,460 (assigned in its entirety to Pacesetter, Inc.), titled “Cardiac Resynchronization Therapy Optimization Using Parameter Estimation from Realtime Electrode Motion Tracking,” the disclosure of which is hereby incorporated by reference.

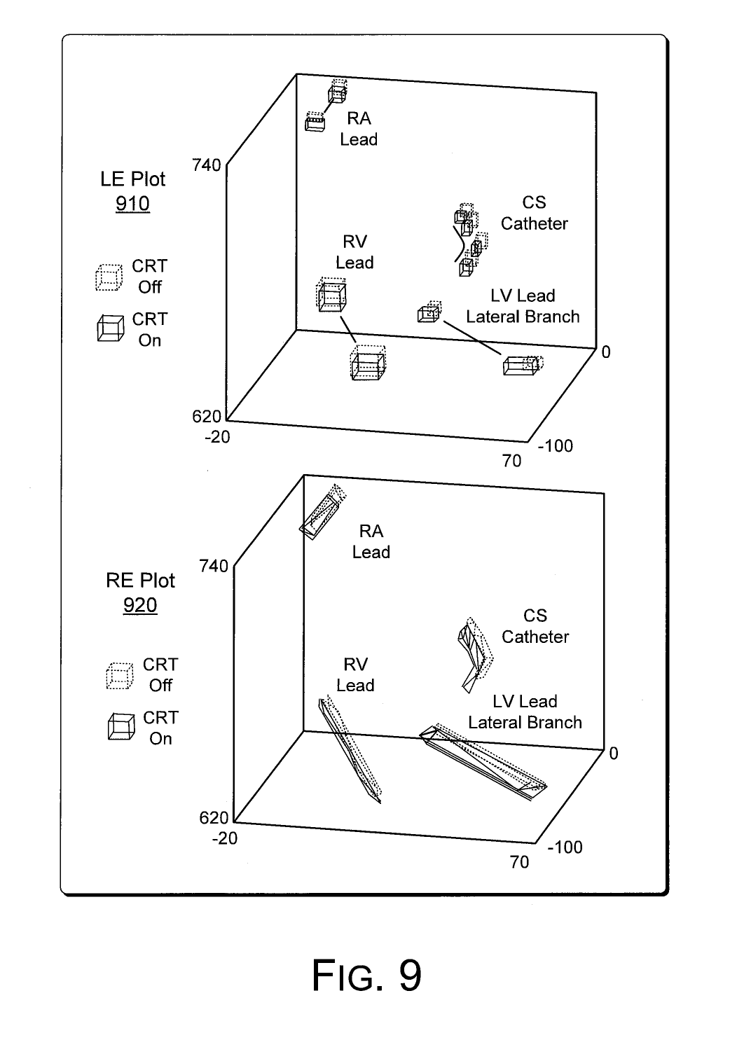

FIG. 9 shows various exemplary volume metrics in three-dimensional plots 910 and 920. In FIG. 9, the plot 910 shows local estimator metrics and the plot 920 shows regional estimator metrics based on trial data. In the plot 910, the local estimator metrics correspond to local volumes defined by movement of an electrode during at least a portion of a cardiac cycle. For example, the RA lead local estimator metrics correspond to coordinates of a tip electrode and a ring electrode for various cardiac cycles. In contrast, for the plot 920, the regional volume estimator metrics correspond to regional volumes defined by movement of electrodes during at least a portion of a cardiac cycle. For example, the RV lead regional estimator metric corresponds to a regional volume defined by coordinates of two electrodes of an RV lead for various cardiac cycles. As described herein, a global volume estimator metric may be defined by various electrodes (e.g., various electrodes can define a left ventricle global volume estimator).

In the acute trial data of FIG. 9, an RA lead, an RV lead, a LV lead, and a coronary sinus catheter were positioned and data acquired for two conditions: CRT off (dashed lines) and CRT on (solid lines). The local estimator plot 910 shows the “volume” of motion of each mapped electrode for both conditions. As indicated at the LV lead, the volume for each of the local estimators with CRT “on” is greater than the volume for each the local estimators with CRT “off”. The 3-D local estimator plot 910 quickly allows a clinician to assess a condition, generally by comparing local estimators. While two conditions may be compared, a comparison may also occur between sites, for example, more movement occurs at the RV lead electrode sites than the CS catheter electrode sites as indicated by the local estimator volume being greater at the RV lead electrode sites than at the CS catheter electrode sites.

The regional estimator plot 920 generally shows volume swept by the respective leads or catheter for the two different conditions (CRT off/on). In the example of FIG. 9, the regional estimator plot 920 allows a clinician to readily assess how regions of the heart may be moving in response to CRT. For example, in response to CRT, one of the LV lead electrodes may move differently in comparison to another one of LV lead electrodes whereas without CRT the two electrodes move similarly. The regional estimators in the plot 920 can indicate such behavior, for example, via conical or other shapes that may exhibit twisting, bending, etc.

As described herein, the information presented in the plots 910 and 920 of FIG. 9 may be alternatively presented as volume waveforms (e.g., where volume is plotted versus time). An exemplary method can acquire position information and calculate volume based on the position information. Such a method can track changes in volume during different pacing interventions and render volume waveforms, which may be viewed by a clinician and further analyzed. For example, an exemplary method can include extracting one or more features from such waveforms. Such features may include systolic slope, diastolic slope, peak-to-peak magnitude, local peak-to-peak, number of peaks, morphological analysis, baseline volume, longitudinal/circumferential fractional shortening, etc. Regional or global dyssynchrony can also be computed from analyzing volumetric changes or waveforms for different pacing interventions.

Further details on volume based metrics are provided in U.S. patent application Ser. No. 12/398,460 (assigned in its entirety to Pacesetter, Inc.), titled “Cardiac Resynchronization Therapy Optimization Using Parameter Estimation from Realtime Electrode Motion Tracking,” the disclosure of which is hereby incorporated by reference.

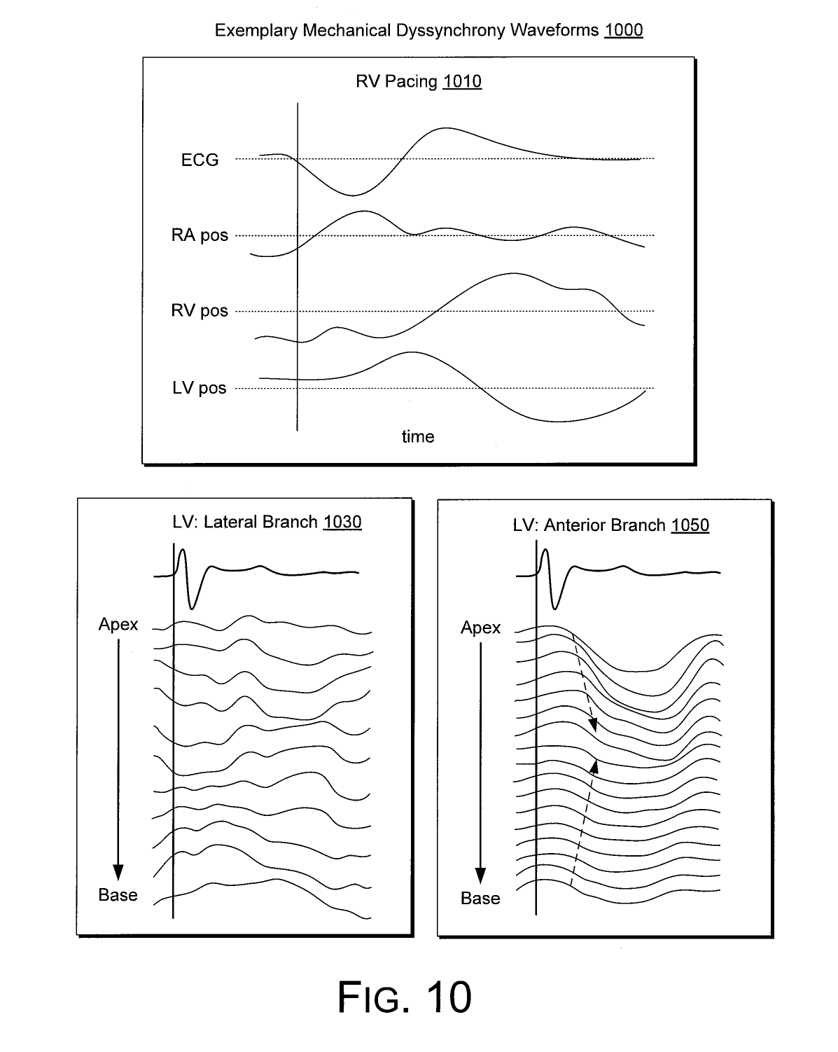

FIG. 10 shows exemplary mechanical dyssynchrony waveforms 1000. In FIG. 10, a plot 1010 shows data acquired during RV pacing. Specifically, the plot 1010 shows an electrogram, position data for an RA electrode, position data for an RV electrode and position data for a LV electrode. During a trial, position data was acquired for two left ventricular electrode sites. A plot 1030 shows position data versus time for a lateral branch of the coronary sinus and a plot 1050 shows position data versus time for an anterior branch of the coronary sinus. The motion waveforms of the plots 1030 and 1050 can be analyzed to uncover mechanical activation patterns.

As described herein, mechanical activation time can be determined from motion waveforms recorded for different regions (e.g., ventricular or atrial). Given waveforms for two regions, a timing based mechanical dyssynchrony metric can be calculated. Referring again to the vectors and relationships of FIG. 6, a mechanical dyssynchrony metric can be calculated for any two vector configurations to provide a local mechanical dyssynchrony metric or between two groups of electrodes for different regions to provide a regional mechanical dyssynchrony metric. As described herein, a mechanical activation time metric can be determined from an analysis of a motion waveform. For example, a mechanical activation time metric may corresponding to onset of inward motion, maximum first negative derivative with respect to time, maximum second negative derivative with respect to time, maximum point (e.g., a diastolic point) or minimum point (e.g., a systolic point).

Further details on mechanical dyssynchrony based metrics are provided in U.S. Patent Application Publication No. 2009/0318995 (assigned in its entirety to Pacesetter, Inc.), titled “Cardiac Resynchronization Therapy Optimization Using Mechanical Dyssynchrony and Shortening Parameters from Realtime Electrode Motion Tracking,” the disclosure of which is hereby incorporated by reference.

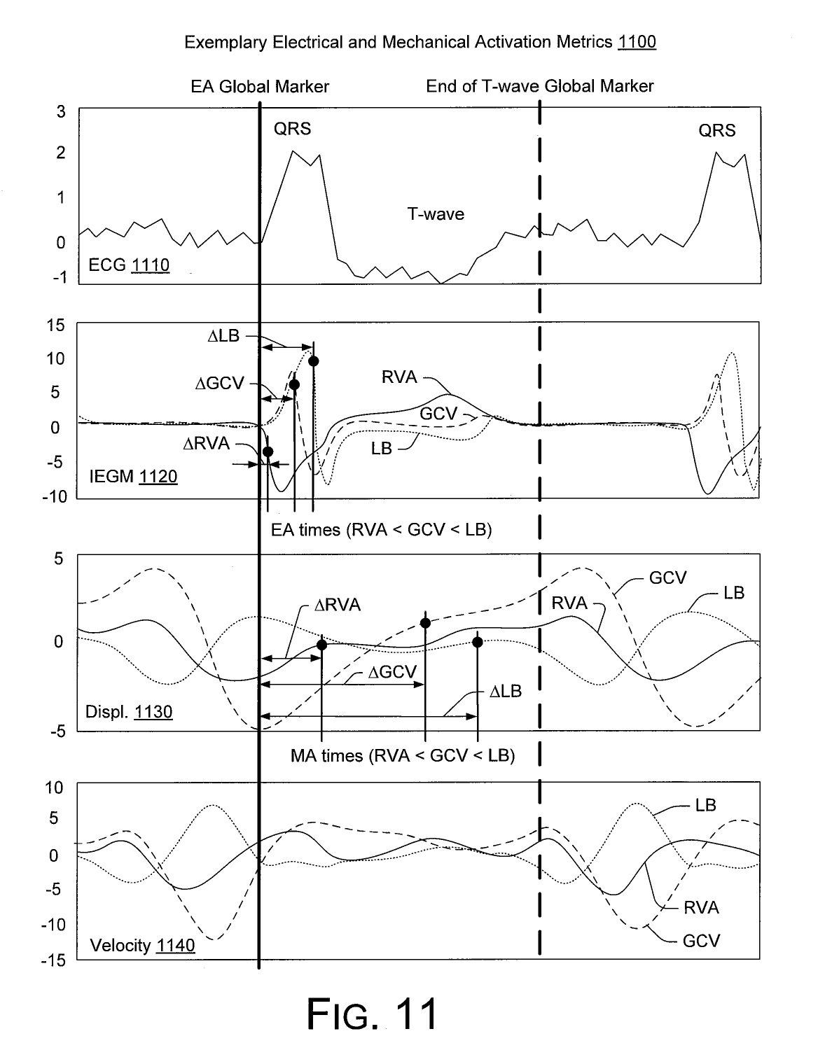

FIG. 11 shows exemplary electrical and mechanical activation metrics 1100. Specifically, FIG. 11 shows a series of plots including an ECG plot 1110, an IEGM plot 1120, a displacement plot 1130 and a velocity plot 1140. The trial data of FIG. 11 was acquired based on spontaneous rhythms (e.g., intrinsic). Configurations included electrode-bearing leads positioned in the RV apex (RVA, solid curve), Great Cardiac Vein (GCV, dashed curve), and Lateral Branch (LB, dotted curve). The ECG plot 1110 data was acquired using surface electrodes and the IEGM plot 1120 data was acquired using unipolar sensing. Data in the plots 1130 and 1140 is discussed further below.

A solid black vertical line extends across all of the plots and represents a time marker for the end diastole, or onset of a global QRS complex (e.g., a global electrical activation time marker). A dashed black vertical line extends across all of the plots and represents a global time marker for the end of the T-wave, which occurs after the end of systole during repolarization.

As described herein, a local electrical delay metric can be identified based on the “global” ECG data and the various “local” IEGM data (electrical information), for example, from onset of the ECG QRS complex to onset of individual local electrical activation as determined by steepest descent of IEGM data acquired for the RVA, GCV and LB. These points in time (electrical activation times, referred to as EA times or EATs) are marked with vertical lines where the local electrical delay metrics are indicated by ΔRVA, ΔGCV and ΔLB as being the measured from the “global” end diastole time to an activation feature of a respective IEGM (e.g., based on steepest or other feature).

As described herein, local mechanical activation metric can be found from peak systolic displacement, which may be defined as the peak of the displacement curve or alternatively as a threshold amplitude (e.g., an inflection point of the displacement curve between onset of a Q-wave to the end of a T-wave). In the displacement plot 1130 of FIG. 11, the mechanical activation times (referred to as MA times or MATs) for the RVA, GCV and LB correspond to inflection points (filled circles) and are indicated with vertical lines.

As shown in FIG. 11, the global QRS complex time from the surface ECG data (plot 1110) can be used to associate the local electrical information (IEGMs of the plot 1120) and the local mechanical information (displacement data of the plot 1130). As mentioned, such association allows for determination of local electromechanical delay metrics (EMDs) as it can provide a common timeline for electrical events and mechanical events. Where a common timeline exists, inherently or by other means, then an association exists upon acquisition of the electrical information and the mechanical information.

For purposes of determination of local electromechanical delays (LEMDs), at each electrode, the time of electrical activation can be determined from “local” EGM data (e.g., detection by peak amplitude, peak negative slope, achieving a threshold voltage or slope, etc.). The electrode position \vec{x}₀ at the time of electrical activation can be noted for one or more electrodes. The electrode position \vec{x}₀ at each subsequent time sample (e.g., until the time of the next detected electrical activation) can be noted, and a distance vector computed as the vector difference between the current position and the position at activation \vec{s}ᵢ=\vec{x}ᵢ−\vec{x}₀.

As shown in FIG. 11, mechanical activation can be determined as the point of inflection, that is, where an increasing rate of change of sᵢ=|sᵢ| reaches a plateau over all points i in a global time window, e.g., time between onset of a Q-wave to the end of a T-wave. In the displacement plot 1130, this is representative of the displacement curve inflection point (filled circles) and indicated by the solid vertical lines for RVA, GCV and LB (MA times). FIG. 11 also indicates that the MA times are as follows: RVA<GCV<LB. In the plot 1130, a slight dip occurs for the LB displacement curve prior to leveling off (i.e., prior to reaching a plateau). To avoid marking a MA time by such a slight dip, an algorithm can determine if rate of change in displacement after a minimum or maximum (e.g., depending on whether displacement is expected to increase or decrease) is less than a predetermined value. For example, in the plot 1130, while actual times in milliseconds are not shown, the trial data indicates that the dip or minimum occurs at about 1700 ms and a plateau occurs in the LB displacement at about 1770 ms and the plateau spans about 100 ms. An algorithm may be programmed to account for particularities of a region of the heart based on a priori knowledge (e.g., knowing that the mechanical activation times occur in a particular order or that a region may exhibit particular displacement morphology).

Further details on electrical and mechanical activation based metrics are provided in U.S. Patent Application Publication No. 2009/0306732 (assigned in its entirety to Pacesetter, Inc.), titled “Cardiac Resynchronization Therapy Optimization Using Electromechanical Delay from Realtime Electrode Motion Tracking,” the disclosure of which is hereby incorporated by reference.

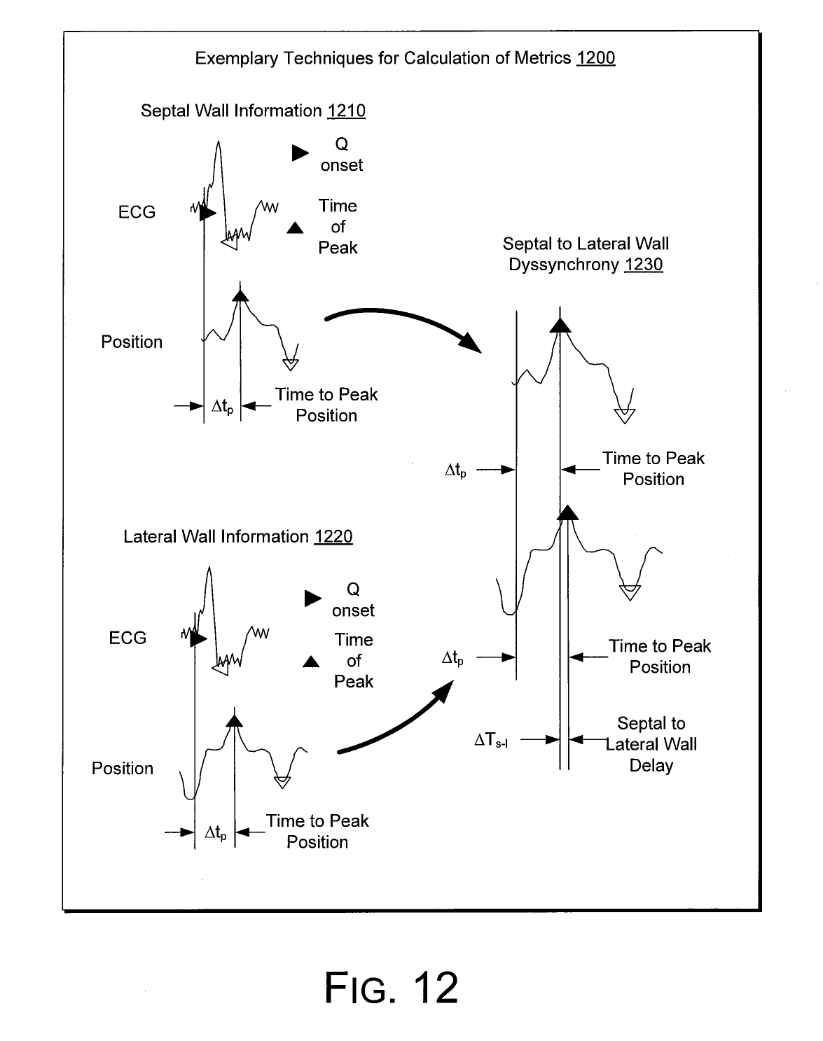

FIG. 12 shows exemplary techniques for calculation of metrics 1200. Specifically, FIG. 12 shows a plot of septal wall information 1210 based on data acquired using a distal tip electrode on an RV apical lead during RV-only pacing. In the plot 1210, an ECG for a single cardiac cycle includes a filled triangle that marks a Q wave onset time and a position waveform for the same cardiac cycle includes a filled triangle that marks a peak position time. As shown, for the septal wall, the difference between these two times is the time to peak position during the cardiac cycle, which is referred to as Δtₚ.

FIG. 12 also shows a plot of LV lateral wall information 1220 based on data acquired using a distal tip electrode on a catheter in a lateral branch vein of the left ventricle during RV-only pacing. In the plot 1220, an ECG for a single cardiac cycle includes a filled triangle that marks a Q wave onset time and a position waveform for the same cardiac cycle includes a filled triangle that marks a peak position time. As shown, for the lateral wall, the difference between these two times is the time to peak position during the cardiac cycle, which is referred to as Δtₚ.

FIG. 12 further shows a plot 1230 of septal wall information and lateral wall information sufficient to calculate a septal-to-lateral wall dyssynchrony metric. Specifically, the plot 1230 shows the difference between Δtₚ of the septal wall and Δtₚ of the lateral wall as a measure of septal wall to lateral wall delay, which is indicative of septal-to-lateral wall dyssynchrony and, in general, ventricular dyssynchrony.

In trial data for right atrial pacing (RA pacing), right ventricular pacing (RV pacing), left ventricular pacing (LV pacing) and biventricular pacing (BiV pacing), calculation of right-to-left dyssynchrony and maximum velocity (change in position with respect to time) demonstrated that RV pacing produced the greatest right-to-left dyssynchrony and the lowest velocity; whereas, LV pacing produced the smallest right-to-left dyssynchrony and the highest velocity. The results also indicated that a particular biventricular pacing configuration performed quite similarly to the tested LV pacing condition.

Further details on mechanical dyssynchrony based metrics are provided in U.S. Patent Application Publication No. 2009/0318995 (assigned in its entirety to Pacesetter, Inc.), titled “Cardiac Resynchronization Therapy Optimization Using Mechanical Dyssynchrony and Shortening Parameters from Realtime Electrode Motion Tracking,” the disclosure of which is hereby incorporated by reference.

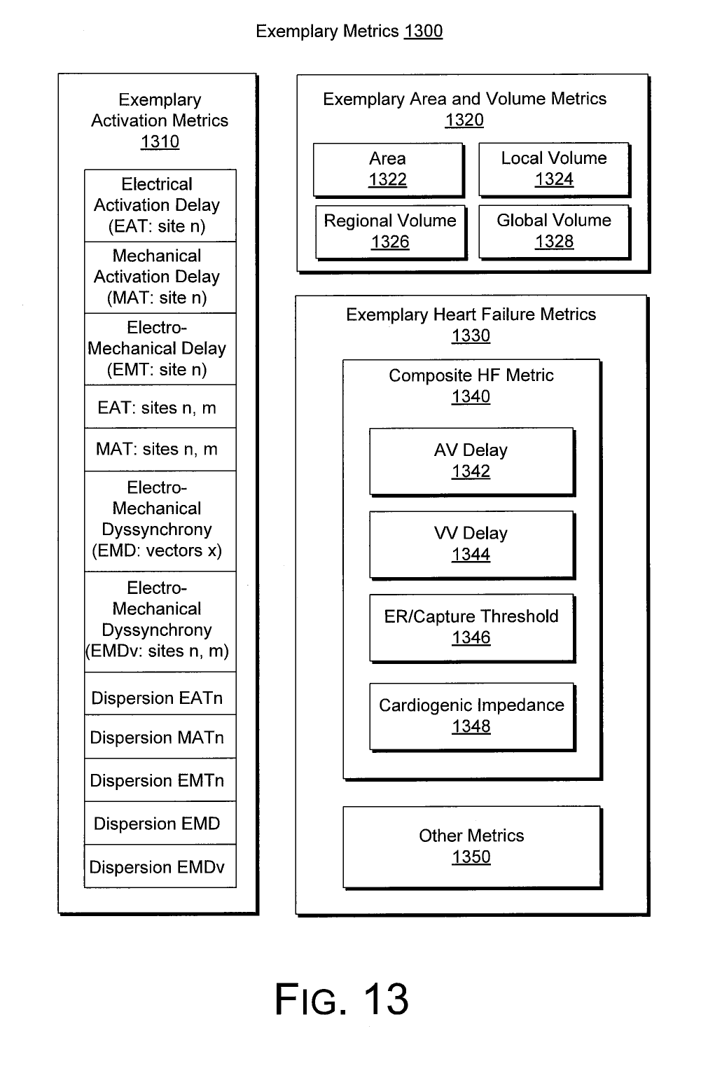

FIG. 13 shows various exemplary metrics 1300, including activation metrics 1310, area and volume metrics 1320 and heart failure metrics 1330. As described herein, a localization system can measure 3-D position of one or more electrodes to provide data for real-time cardiac performance metrics (e.g., contractility, dyssynchrony, volumetric, etc.). Such metrics can facilitate optimization of a pacing therapy, especially CRT. Such metrics can particularly facilitate optimization in an acute phase (e.g., at time of implantation of a CRT device). A method referred to at times as “QuickStim”, involves pacing optimization, for example, to find a optimal single or optimal multisite pacing settings for implantable pacing devices, especially those with multiple LV pacing capability and a multipolar LV lead. Such a method may be automated or semi-automated.

An exemplary QuickStim method may provide for optimization of a pacing site, a pacing vector configuration, that may include a single-site LV electrode configuration in combination with the device housing or other electrodes elsewhere in the body, or a multi-site LV electrode configuration either alone or in combination with other electrodes elsewhere in the body. Such a method can involve real-time data acquisition or monitoring of electrical and hemodynamic responses to trial configurations. For example, the aforementioned ENSITE® NAVX® localization system may be used to acquire and optionally analyze such data using specialized modules that implement one or more exemplary methods described herein. Alternatively, data acquired may be transferred to another computing device for analysis.

As mentioned, in addition to position data (e.g., position versus time or motion data), electrical data can be obtained. As explained, electromechanical dyssynchrony metrics can be determined based on an analysis of the distribution of electromechanical delays over a region (e.g., including myocardium of both ventricles). An exemplary method may include mapping of electromechanical delay metrics at multiple sites along with local or regional electromechanical dyssynchrony metrics, for example, calculated based on analysis of differences in electromechanical delay between two or more electrodes or sites (see, e.g., vector configurations of FIG. 6). As described herein, spatial and temporal dispersions metrics of electrical activation delays and mechanical activation delays or electromechanical delay can also be determined (e.g., for various vectors).

Referring again to the exemplary metrics 1300 of FIG. 13, the action metrics 1310 pertain to action of the heart over a cardiac cycle, particular key times where electrical activity occurs or where mechanical activity occurs. The examples of activation metrics 1310 in FIG. 13 include: electrical activation delay at a site n (EATn; see, e.g., FIG. 11); mechanical activation delay at a site n (MATn; see, e.g., FIG. 11); electromechanical delay at a site n (e.g., EMTn=abs(EATn−MATn); see, e.g., FIG. 12); electrical activation delay between two sites, n and m (e.g., as a vector, EATnm); mechanical activation delay between two sites, n and m (e.g., as a vector, MATnm); electromechanical dyssynchrony (e.g., where EMD=EMTn−EMTn+1 . . . x, where x=total number of available vectors); electromechanical dyssynchrony between two sites, n and m (e.g., as a vector, EMDv=abs(EATnm-MATnm)); dispersion of EATn (e.g., stdev(EATn . . . EATn+x)); dispersion of MATn (e.g., stdev(MATn . . . MATn+x)); dispersion of EMTn (e.g., stdev(EMTn . . . EMTn+x)); dispersion of EMD and dispersion of EMDv.

The exemplary area and volume metrics 1320 of FIG. 13 include area metrics 1322 (see, e.g., FIG. 8), local volume metrics 1324 (see, e.g., FIG. 9), regional volume metrics 1326 (see, e.g., FIG. 9) and global volume metrics 1328.

The exemplary heart failure metrics 1330 of FIG. 13 include a composite heart failure metric 1340 and one or more other metrics 1350. In the example of FIG. 13, the composite heart failure metric 1340 is based on AV delay 1342, W delay 1344, ER or capture threshold 1346 and cardiogenic impedance 1348. Based on these four factors, a metric can be calculated that provides an indication of heart failure, particularly worsening heart failure. The underlying factors for the metric 1340 may optionally be determined by an implantable pacing device. For example, an exemplary pacing device may include an algorithm that receives AV delay, VV delay, capture threshold and cardiogenic impedance values and outputs a value for a heart failure metric. In turn, this metric may trigger an alert, an optimization algorithm or both, especially when the value indicates that heart failure is worsening (e.g., by comparison to a predetermined value or one or more historic values). The one or more other metrics 1350 may be based on any of a variety of factors. For example, an oxygen sensor, an accelerometer, or other sensor may provide information sufficient for a metric indicative of heart failure or a change in heart failure status of a patient.

While FIG. 13 presents some metrics, as mentioned, yet others may be used. Details on IEGM metrics corresponding to myocardial infarction and scarring are provided in U.S. patent application Ser. No. 12/639,788 (assigned in its entirety to Pacesetter, Inc.), titled “Methods to Identify Damaged or Scarred Tissue Based on Position Information and Physiological Information,” the disclosure of which is hereby incorporated by reference. Details on energy drain metrics corresponding to myocardial infarction and scarring are provided in U.S. patent application Ser. No. 12/553,413 (assigned in its entirety to Pacesetter, Inc.), titled “Pacing, Sensing and Other Parameter Maps Based on Localization System Data,” the disclosure of which is hereby incorporated by reference. Details on stability metrics corresponding to myocardial infarction and scarring are provided in U.S. patent application Ser. No. 12/562,003 (assigned in its entirety to Pacesetter, Inc.), titled “Electrode and Lead Stability Indexes and Stability Maps Based on Localization System Data,” the disclosure of which is hereby incorporated by reference.

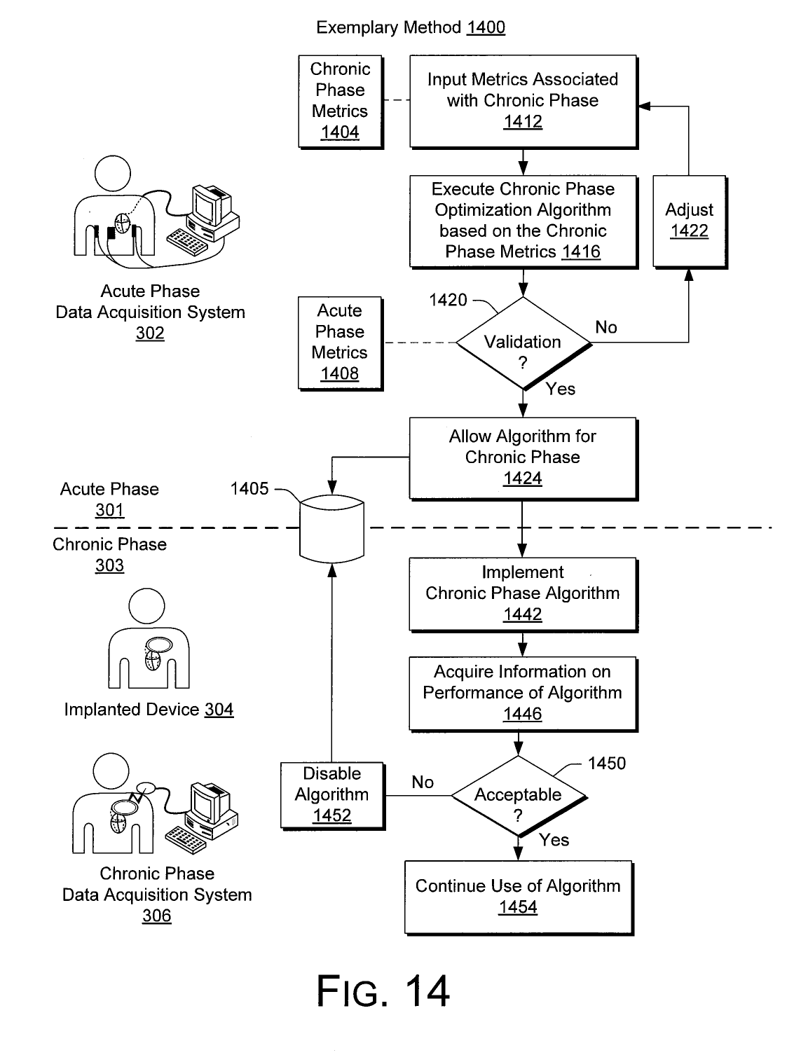

FIG. 14 shows an exemplary method 1400 for validating and implementing a chronic phase algorithm for optimizing pacing configuration of an implantable pacing device. FIG. 14 shows the equipment 302, 304 and 306 of FIG. 3 as well as the acute phase 301 and the chronic phase 303. As described herein, various actions occur in the acute phase 301 to validate an algorithm for use in the chronic phase 303. As explained, optimization in the chronic phase 303 may be limited as to the types of metrics available.

The method 1400 includes chronic phase metrics 1404 and acute phase metrics 1408 where the acute phase metrics 1408 include position-based metrics such as the activation metrics 1410 and area and volume metrics 1420 of FIG. 13. The chronic phase metrics 1404, typically, do not include position-based metrics such as those based on information acquired by the ENSITE® NAVX® localization system and instead are typically limited to IEGM-based metrics such as AV delay, VV delay, ER/capture threshold and impedance-based metrics, such as cardiogenic impedance. In scenarios where some position-based metrics are available in the chronic phase 303, the metrics may be limited, for example, as physical repositioning of electrodes and leads is typically not possible.

According to the example of FIG. 14, the method 1400 commences in an input block 1412 that receives a listing of metrics associated with a chronic phase. For example, during an acute phase procedures, e.g., device implant, a clinician may enter the type of implantable device 304 and optionally associated programming device 306 in a graphical user interface (GUI), which may be part of the acute phase data acquisition system 302. The method 1400 may then access a database within the acute phase data acquisition system 302 that stores a listing of metrics 1404 available for that implantable device 304. For example, a device's chronic phase metrics may include IEGM-based metrics such as AV delay, VV delay, ER/capture threshold and impedance-based metrics such as cardiogenic impedance. Given the chronic phase metrics 1404 associated with the implantable device, in an execution block 1416, the method 1400 executes a chronic phase optimization algorithm that relies on, or is based on, one or more of the available chronic phase metrics 1404. For example, for a device having one or both of IEGM-based chronic phase metrics and impedance-based metrics, multiple LV pacing capability and a multipolar LV lead, a QuickStim algorithm for optimizing pacing site, pacing electrode configuration, number of LV pacing electrodes, and/or pacing parameters, such as AV delay and VV delay may be executed. As mentioned, chronic phase metrics 1404 are typically limited to those that can be determined based on information acquired by an implantable device during a chronic phase. In some instances, information may be acquired (e.g., by an external device at a follow-up visit) and communicated to an implantable device for use in a chronic phase optimization algorithm.

During or after execution of the chronic phase optimization algorithm, the method 1400 enters a validation block 1420 that decides if the chronic phase optimization algorithm can be validated for use in the chronic phase 303. In general, this validation 1420 involves a comparison of device configuration selections (e.g., electrode vector configuration, pacing parameters, etc.) made by the chronic phase optimization algorithm, with like device configuration selections made based on acute phase metrics acquired by the acute phase data acquisition system. The chronic phase optimization algorithm is validated if there is some level of agreement between the respective selections. As shown in the example of FIG. 14, the validation block 1420 relies on acute phase metrics 1408, which can provide a more detailed indication of cardiac performance. As part of the validation block 202, the acute phase data acquisition system 302 acquires acute phase information that allows for the calculation and determination of one or more acute phase metrics 1408. For example, the acute phase data acquisition system 302 may include the ENSITE® NAVX® system, which as previously described, allows for the acquisition of electrode position information with respect to time, which in turn allows for the determination of one or more of previously described motion-based metrics, vector-based metrics, area-based metrics, volume-based metrics and/or heart failure metrics. Where the chronic phase optimization algorithm selects a configuration (electrode vector configuration, pacing parameters, etc.) as being optimal, the acute phase metrics 1408 determined by the acute phase data acquisition system 302 may be analyzed to decide if the configuration selected by the chronic phase optimization algorithm is optimal or at least near optimal and not sub-optimal. For example, an acute phase optimization algorithm may be configured to rank electrode configurations based on acute phase metrics, which as noted previously, tend to be more accurate measures of cardiac performance/CRT performance compared to chronic phase metrics. In this example, the validation block 1420 compares an optimal electrode configuration of the chronic phase optimization algorithm to the ranked configurations. If the optimal electrode configuration does not match the top ranked configuration, then the validation block 1420 will compare it to the next highest ranked electrode configuration until a match occurs. Where no exact match occurs, the validation block 1420 may indicate closeness, partial match, etc., based on configuration parameters.

As to a partial match, if three of four electrodes of the two configurations match, a 75% match indicator may be displayed. Such a match value may be considered sufficient to validate the chronic phase optimization algorithm. As to closeness, in the foregoing example, where the non-matching electrode is a neighboring electrode (e.g., in a series of LV electrodes), a closeness indicator may be displayed. Such a method may indicate a neighboring electrode as “−1”, a next nearest neighboring electrode as “−2”, etc. As described herein, in another example, a chronic phase optimization algorithm may output a ranked list of configurations (chronic phase configurations or “CPC”s) and these may be compared to a ranked list of configurations based on acute phase metrics (acute phase configurations or “APC”s). Given two ranked lists, a percentage match (e.g., CPC1=APC1, CPC2=APC2, CPC3≠APC3, etc.) or offset (e.g., CPC3=APC4, offset=1) may be determined and relied upon to determine whether the chronic phase optimization algorithm can be validated. In the foregoing examples, a clinician may assign one or more criteria for validation (e.g., exactness of match, ranking, etc.).

As described herein, a method can include comparing a selected optimal electrode configuration to one or more configurations determined by an acute phase optimization algorithm. Such a comparison can include determining match metric (e.g., match percentage, closeness, etc.). For example, a match metric may be a percentage based on a number of electrodes in a configuration and a number of electrode matches between a selected optimal electrode configuration and another configuration (e.g., as based on acute phase metrics). As mentioned, a method can include executing a chronic phase optimization algorithm to provide a ranked list of electrode configurations based on metrics available as input to the chronic phase optimization algorithm. In such an example, a method can include validating by comparing the ranked list of electrode configurations to a ranked list of configurations determined by an acute phase optimization algorithm based on metrics available as input to the acute phase optimization algorithm.

Referring again to FIG. 14, if the validation block 1420 decides that the algorithm is valid, the method 1400 continues to an allowance block 1424 that allows the chronic phase optimization algorithm to be used for the chronic phase 303. However, if the validation block 1420 fails to validate the algorithm, the method 1400 continues at an adjustment block 1422 that may adjust the chronic phase optimization algorithm, which may include excluding or including certain chronic phase metrics that may improve probability of validation in a subsequent loop. Alternatively, the method 1400 may terminate and not allow implementation of the chronic phase optimization algorithm for the chronic phase 303 and optionally allow for programming of the device to the configuration determined by the acute phase optimization algorithm. Similarly, the method 1400 may terminate if the chronic phase optimization algorithm cannot be validated after a number of adjustment loops.

Where the method 1400 allows for implementation of the algorithm in the chronic phase 303, information associated with the validation process may be sent to a data storage 1405. As described herein, the data storage 1405 may be accessible via a network and store information for multiple patients (e.g., acute phase and chronic phase information).

Upon validation, the algorithm is implemented in an implementation block 1442 in the chronic phase 303. According to the example of FIG. 14, the method 1400 includes an acquisition block 1446 that acquires or monitors information associated with performance of the algorithm in the chronic phase 303. Further, a decision block 1450 decides if the performance is acceptable. Such a decision may be made automatically by the implanted device 304 or it may be made in conjunction with intervention by a chronic phase data acquisition system 306 (e.g., a programming device or programmer). If the decision block 1450 decides that the performance of the chronic phase optimization algorithm is acceptable, the method 1400 proceeds to a continuation block 1454 where the implantable device 304 continues to use the algorithm, including operating automatically once the patient leaves the follow-up clinic. However, if the decision block 1450 decides that the performance of the chronic phase optimization algorithm is not acceptable, the method 1400 continues at a disable block 1452 that disables the algorithm. Such a decision may be recorded and stored in the data storage 1405, optionally with information upon which the decision was based. In subsequent procedures, such information may be relied upon in an acute phase to decide whether an algorithm is valid.

As mentioned, the ENSITE® NAVX® localization and mapping system provides for tracking three-dimensional positions of electrodes and mapping such positions with respect to one or more anatomical markers (e.g., of cardiac structure). Further, the ENSITE® NAVX® system can record electrical activity at various electrode locations. In combination, such information provides indications of real-time cardiac performance that, as described herein, can be used in an exemplary method to validate an optimization algorithm (e.g., a “QuickStim” algorithm). Such an algorithm may be implemented in a chronic phase by an implantable device alone or in conjunction with an external device (e.g., a programmer). As described herein, an exemplary optimization algorithm for chronic phase use can determine optimal single or multisite pacing settings for an implantable device.

### CONCLUSION

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A method comprising:
based on metrics available as input to a chronic phase optimization algorithm for selecting an optimal electrode configuration for delivery of a cardiac pacing therapy, executing the chronic phase optimization algorithm during an acute phase to select an optimal electrode configuration for delivery of a cardiac pacing therapy;
during the acute phase, acquiring position information with respect to time for electrodes implanted in a body;
determining one or more acute phase metrics based on the acquired position information; and
validating the chronic phase optimization algorithm based at least in part on the one or more acute phase metrics.

2. The method of claim 1 wherein the metrics available as input to the chronic phase optimization algorithm comprise one or more IEGM-based metrics.

3. The method of claim 1 wherein the metrics available as input to the chronic phase optimization algorithm comprise one or more impedance-based metrics.

4. The method of claim 1 wherein the metrics available as input to the chronic phase optimization algorithm comprise one or more vector-based metrics.

5. The method of claim 1 wherein the acute phase metrics comprise one or more motion-based metrics.

6. The method of claim 1 wherein the acute phase metrics comprise one or more vector-based metrics.

7. The method of claim 1 wherein the acute phase metrics comprise one or more area-based metrics.

8. The method of claim 1 wherein the acute phase metrics comprise one or more volume-based metrics.

9. The method of claim 1 wherein the metrics comprise a heart failure metric based at least in part on one or more interchamber delays.

10. The method of claim 1 wherein the validating comprises executing an acute phase optimization algorithm to select an optimal electrode configuration for delivery of the cardiac pacing therapy and comparing the optimal electrode configuration selected by the acute phase optimization algorithm with the optimal electrode configuration selected by the chronic phase optimization algorithm.

11. The method of claim 1 further comprising enabling the chronic phase optimization algorithm for implementation during a chronic phase.

12. The method of claim 11 further comprising assessing performance of the chronic phase optimization algorithm during the chronic phase based at least in part on the one or more acute phase metrics.

13. The method of claim 12 further comprising deciding whether to disable the chronic phase optimization algorithm based on the assessing performance.

14. The method of claim 1 further comprising storing one or more metrics to a storage device.

15. The method of claim 1 wherein the validating comprises comparing the selected optimal electrode configuration to one or more configurations determined by an acute phase optimization algorithm.

16. The method of claim 15 further comprising determining a match metric based on the comparing.

17. The method of claim 16 wherein the match metric comprises a percentage based on a number of electrodes in a configuration and a number of electrode matches between the selected optimal electrode configuration and one of the one or more configurations.

18. The method of claim 1 further comprising executing the chronic phase optimization algorithm to provide a ranked list of electrode configurations based on metrics available as input to the chronic phase optimization algorithm.

19. The method of claim 18 wherein the validating comprises comparing the ranked list of electrode configurations to a ranked list of configurations determined by an acute phase optimization algorithm based on metrics available as input to the acute phase optimization algorithm.

20. The method of claim 1 wherein selecting the optimal electrode configuration comprises selecting a pacing vector configuration that may include a single-site LV electrode configuration in combination with the device housing or other electrodes elsewhere in the body, or a multi-site LV electrode configuration either alone or in combination with other electrodes elsewhere in the body.

21. The method of claim 1, wherein acquiring position information with respect to time for electrodes implanted in a body comprises acquiring such information during delivery of a cardiac pacing therapy corresponding to the cardiac pacing therapy for which the chronic phase optimization algorithm selected an optimal electrode configuration.
